(19) United States
(12) Patent Application Publication
(10) Pub. No.: US 2011/0301192 A1
(43) Pub. Date: Dec. 8, 2011

(54) Inhibitors of Cyclin Kinase Inhibitor p21

(75) Inventors: Robert Weiss, Vacaville, CA (US); See-Hyoung Park, Palo Alto, CA (US); Kit S. Lam, Davis, CA (US); Ruiwu Liu, Sacramento, CA (US)

(73) Assignee: The regents of the University of California, Oakland, CA (US)

(21) Appl. No.: 13/119,152

(22) PCT Filed: Sep. 29, 2009
(86) PCT No.: PCT/US09/58697

§ 371 Date: Aug. 5, 2011

Related U.S. Application Data
(60) Provisional application No. 61/101,963, filed on Oct. 1, 2008.
(60) Provisional application No. 61/150,404, filed on Feb. 6, 2009.
(60) Provisional application No. 61/237,356, filed on Aug. 27, 2009.

(51) Int. Cl. A61K 31/454; C07D 235/18; C07D 403/06; A61P 35/00; C07D 401/04; A61K 31/4184; A61K 31/4375; C07D 401/06; C07D 455/02

(52) U.S. Cl. 514/306; 546/199; 548/309.7; 548/306.1; 546/138; 514/322; 514/394

(57) ABSTRACT

The present invention provides compounds that inhibit cyclin kinase inhibitor p21, such as compounds of formula I. The present invention also provides compositions including compounds of Formula I and a pharmaceutically acceptable excipient. In addition, the present invention provides methods of inhibiting cyclin kinase inhibitor p21 and of treating cancer.

### CROSS-REFERENCES TO RELATED APPLICATIONS

This application claims priority to U.S. Provisional Application Nos. 61/237,356, filed Aug. 27, 2009, 61/150,404, filed Feb. 6, 2009, and 61/101,963, filed Oct. 1, 2008, which are incorporated in their entirety herein for all purposes.

### STATEMENT AS TO RIGHTS TO INVENTIONS MADE UNDER FEDERALLY SPONSORED RESEARCH AND DEVELOPMENT

This invention was made with Government support under Grant Nos. U01CA084986 and R01CA098116, awarded by the National Institutes of Health. The Government has certain rights in this invention.

### BACKGROUND OF THE INVENTION

Kidney cancer (or renal cell carcinoma; RCC) is responsible for 13,000 deaths annually in the US. The disease is frequently asymptomatic, and a third of cases are diagnosed when the disease is already metastatic, at which time it has 95% mortality (Weiss R H, Lin P-Y. Kidney Int 2006; 69(2):224-232). Conventional treatment of RCC has been based on surgical approaches and the administration of the immunomodulating medications interferon and interleukin-2 (reviewed in Weiss R H, Lin P-Y. Kidney Int 2006; 69(2):224-232). These drugs, their combinations with cytotoxic “standard” treatments such as gemcitabine and 5-FU (Stadler W M, Huo D, George C, Yang X, Ryan C W, Karrison T et al. J Urol 2003; 170(4 Pt 1):1141-1145), other cytostatic chemotherapy agents alone and in combination, as well as hormonal manipulation, have all shown no benefit to survival and are thus not generally utilized (reviewed in De Mulder P H, van Herpen C M, Mulders P A Ann Oncol 2004; 15 Suppl 4:iv319-iv328). Novel targeted therapies are just beginning to emerge, with the most promising being the kinase inhibitors sorafenib and sunitinib (reviewed in ref Tuma R S. J Natl Cancer Inst 2004; 96(17):1270-1271). However, new pharmacological approaches which would cause standard therapies to be effective in this disease would be a welcome addition to the limited available armamentarium.

p21 is a member of the cip/kip family of cyclin kinase “inhibitors,” but this protein also possesses a variety of properties relating to apoptosis (Matsushita H, Morishita R, Kida I, Aoki M, Hayashi S, Tomita N et al. Hypertension 1998; 31:493-498; Asada M, Yamada T, Ichijo H, Delia D, Miyazono K, Fukumuro K et al. EMBO J 1999; 18(5):1223-1234; Tian H, Wittmack E K, Jorgensen T J. Cancer Res 2000 Feb. 1; 60 (3):679-84 2000; 60:679-684; Fan Y, Borowsky A D, Weiss R H. Mol Cancer Ther 2003; 2(8):773-782) as well as cell proliferation (Weiss R H, Joo A, Randour C. J Biol Chem 2000; 275:10285-10290; Kavurma M K, Khachigian L M. J Biol Chem 2003; 278:32537-32543; Dong Y, Chi S L, Borowsky A D, Fan Y, Weiss R H. Cell Signal 2003; 16(2):263-269). The initial descriptions of p21 focused on its location in the tumor suppressor pathway downstream of p53 (el-Deiry W S, Tokino T, Velculescu V E, Levy D B, Parsons R, Trent J M et al. Cell 1993; 75:817-825), its function as an inhibitor of G₁ cyclin kinases (Xiong Y, Hannon G J, Zhang H, Casso D, Kobayashi R, Beach D. Nature 1993; 366(6456):701-704; Harper J W, Adami G R, Wei N, Keyomarsi K, Elledge S J. Cell 1993; 75(4):805-816), and its role in differentiation (Sherr C J, Roberts J M. Genes and Dev 1999; 13:1501-1512). However, more recent investigations have shown that p21 also plays roles in allowing cell cycle transit as well as preventing apoptosis (Fan Y P, Weiss R H. J Am Soc Nephrol 2004; 15(3):575-584; Liu X F, Xia Y F, Li M Z, Wang H M, He Y X, Zheng M L et al. Cell Biol Int 2006; 30(3):283-287; Sohn D, Essmann F, Schulze-Osthoff K, Janicke R U. Cancer Res 2006; 66(23):11254-11262; Park S H, Park J Y, Weiss R H. J Urol 2008; 180(1):352-360); since programmed cell death is the ultimate mechanism by which cancer chemotherapeutics exert their salutary effects on tumor cells, this property of p21 has considerable untapped potential to be of fundamental importance in the therapy of human cancer.

For many cancers, treatment with DNA damaging agents, at doses required for efficacy, are associated with unacceptable adverse effects as well as inadequate cure rates. Kidney cancer is notoriously chemotherapy as well as “conventional” immunotherapy resistant, although recent work with kinase inhibitors has shown promise for late-stage disease. A possible reason for chemotherapy resistance is failure of these agents, when used alone, to cause cancer cell apoptosis, since inactivation of apoptosis is essential for cancer development (Brown J M, Attardi L D. Nat Rev Cancer 2005; 5(3):231-237; Evan G I, Vousden K H. Nature 2001; 411(6835):342-348).

In breast cancer, increased cytosolic p21 or higher (total) p21 expression by immunostaining have been linked to poorer prognosis (Winters Z E, Leek R D, Bradburn M J, Norbury C J, Harris A L. Breast Cancer Res 2003; 5(6):242-249), and efforts to attenuate p21 in vitro in breast cancer, and in vivo in breast, colon and esophageal cancers, have lead to salutary effects on tumors cells. In kidney cancer, p21 has been shown to have prognostic value in the clear cell variety which is a function of whether patients have localized or metastatic disease at diagnosis (Weiss R H, Borowsky A D, Seligson D, Lin P Y, Dillard-Telm L, Belldegrun A S et al. J Urol 2007; 177(1):63-68). The likelihood that p21 is preventing cells from undergoing apoptosis and thereby allowing their escape from chemotherapy is supported by the finding that antisense oligodeoxynucleotides in vitro cause kidney cancer cells to be sensitized to DNA-damaging therapy, consistent with the anti-apoptotic effect of p21 observed in other cell lines (Asada M, Yamada T, Ichijo H, Delia D, Miyazono K, Fukumuro K et al. EMBO J 1999; 18(5):1223-1234).

Surprisingly, rather than causing inhibition of p21 activity, the compounds of the present invention induce ubiquitinylation and proteosomal degradation of p21, and consequent sensitization of chemotherapy-induced apoptosis in two RCC cell lines.

### BRIEF SUMMARY OF THE INVENTION

In one embodiment, the present invention provides a compound of formula I:

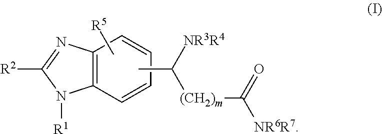

In Formula I, radical R¹ is C₁₋₆ alkyl, C₀₋₆ alkyl-NR⁸R⁹, C₀₋₆ alkyl-cycloalkyl, C₀₋₆ alkyl-heterocycloalkyl, C₀₋₆ alkyl-aryl and C₀₋₆ alkyl-heteroaryl, each optionally substituted with from 1-4 R^{1a} groups. Within R¹, each radical R^{1a} is independently H, halogen, C₁₋₆ alkyl, C₁₋₆ haloalkyl, C₂₋₆ alkenyl, C₂₋₆ alkynyl, C₁₋₆ alkoxy, C₁₋₆ haloalkoxy, C₁₋₆ hydroxyalkyl, —OH, —C₀₋₆ alkyl-NR⁸R⁹, —SR⁸, —C(O)R⁸, —C₀₋₆ alkyl-C(O)OR⁸, —C(O)NR⁸R⁹, —N(R⁸)C(O)R⁹, —N(R⁸)C(O)OR⁹, —N(R⁸)C(O)NR⁸R⁹, —OP(O)(OR⁸)₂, —S(O)₂OR⁸, —S(O)₂NR⁸R⁹, —CN, C₀₋₆ alkyl-cycloalkyl, heterocycloalkyl, C₀₋₆ alkyl-aryl and heteroaryl, alternatively, two R^{1a} groups are joined to form ═O. In Formula I, radical R² is C₀₋₆ alkyl-aryl, C₂₋₆ alkenyl-aryl, C₀₋₆ alkyl-heteroaryl, and —N(R⁸)-aryl, each optionally substituted with 1-4 members that are each independently R^{1a}, —OR¹⁰, —SR¹⁰ or —NR⁸R¹⁰. Radical R³ of Formula I is —C(O)—C₁₋₆ alkyl, —C(O)—C₁₋₆ hydroxyalkyl, —C(O)—C₁₋₆ alkylamine, —C(O)-heterocycloalkyl, —C(O)—NR^{3a}R^{3b}, —C(O)OR^{3a}, —S(O)₂R^{3a} or an amino acid. Within R³, each of radicals R^{3a} and R^{3b} are independently H, C₁₋₆ alkyl, cycloalkyl, heterocycloalkyl, aryl, or heteroaryl, each optionally substituted with from 1-4 R⁸ groups. Radicals R⁴, R⁵, R⁶ and R⁷ of Formula I, as well as radical R⁸ of R^{1a}, R^{3a} and R^{3b}, and radical R⁹ of R^{1a}, are independently H or C₁₋₆ alkyl. Radical R¹⁰ of Formula I is cycloalkyl, heterocycloalkyl, aryl or heteroaryl, each optionally substituted with from 1-4 R^{1a} groups. Subscript m of Formula I is an integer from 0-2. The present invention also includes the salts, hydrates, prodrugs, and isomers of the compounds of Formula I.

In other embodiments, the present invention provides a pharmaceutical composition including a compound of formula I and a pharmaceutically acceptable excipient.

In some other embodiments, the present invention provides a method of inhibiting cyclin kinase inhibitor p21 including administering to a subject in need thereof a therapeutically effective amount of a compound of formula I.

In still other embodiments, the present invention provides a method of treating cancer, the method including administering to a subject in need thereof a therapeutically effective amount of a compound of formula I.

### BRIEF DESCRIPTION OF THE DRAWINGS

FIG. 1 shows a synthetic scheme of a MS-encoded benzoimidazole-based OBOC library.

FIG. 2 shows a method for screening for small molecule ligands to p21 using recombinant p21-GST. OBOC beads tethered with small molecule ligands were incubated with recombinant GST-p21 (or GST alone as control) and incubated with anti-GST antibodies conjugated with alkaline phosphatase. Blue beads in stage 2 were discarded and the true positive beads (i.e., clear beads in stage 2 which indicate binding only to p21) were picked up and submitted for MS decoding.

FIG. 3 shows compounds 4, 7, and 10 decreasing cell viability in two RCC cell lines. ACHN and A-498 cells were incubated in serum-free media for 16 hr and treated with vehicle (DMSO) and serum-containing complete media (CM) alone or the indicated synthesized compounds 4, 7, or 10 (at 10, 50, and 100 μM) in CM. After 48 hr, the MTT assay was performed as described below. The visible absorbance of each well was quantified using a microplate reader.

FIGS. 4a and 4b show p21-binding molecules specifically attenuate p21 levels and cause apoptosis. FIG. 4a shows ACHN cells were incubated in serum-free quiescence media (Q) for 16 hrs and treated with vehicle (DMSO), serum-free media or PDGF (P) and the indicated synthesized compounds 4, 7, or 10 (10, 50, and 100 μM) in serum-free media for 2 hr. Cells were harvested and immunoblotted with p57, p27, and p21. Lane Q: cells incubated in serum-free media alone; lane P: cells incubated with PDGF only. FIG. 4b shows cells incubated in serum-free media for 16 hrs and treated with vehicle (DMSO, CM) indicated synthesized compounds 4, 7, or 10 (at concentrations indicated) in serum-free media for 24 h (top) or 24, 48, and 72 hr (100 μM, bottom). Cells were harvested and immunoblotted with PARP and p21. β-actin is a gel-loading control.

FIG. 5 shows compound 10 decreases p21 stability via a proteasome-mediated pathway. ACHN and A-498 cells were incubated in serum-free media for 16 hr and treated with (a) compound 10 (100 μM), cycloheximide (CHX, 10 μg/ml), or compound 10 plus CHX for the indicated time, or (b) three different proteasome inhibitors (lactascystin, 20 μM; LLnL, 20 μM; MG132, 20 μM) for 6 h and followed by incubation of compound 10 (100 μM) for the indicated time. The density ratio of p21/actin is shown. Q refers to cells incubated in serum-free media only; P refers to cells incubated with PDGF only.

FIG. 6 shows compound 10 causes ubiquitinylation of p21. ACHN and A-498 cells were incubated in serum-free media for 16 hr and treated or not treated with compound 10 (100 μM) for the indicated times. Cells were harvested and immunoprecipitated with p21 antibody-agarose conjugate and immuno-blotted with either p21 or ubiquitin. Ponceau-staining heavy chain of anti-p21-IgG was performed on the same lysate as loading control.

FIG. 7 shows compound 10 causes synergistic anti-proliferative activity and apoptosis when incubated with doxorubicin. ACHN and A-498 cells were incubated in serum-free media for 16 hrs and treated with vehicle (DMSO) or compound 10 (100 μM). After 6 h, doxorubicin was added (0.1 and 0.25 μM) in serum containing media followed in 42 h by (a) the MTT assay as described below; and (b) immunoblotting with PARP, p21, and β-actin.

FIG. 8 shows compounds LLW15, LLW33, LLW36, LLW38, LLW39, LLW40 and LLW42 decreasing cell viability in kidney cancer cell lines. The kidney cancer cells were incubated in serum-free media for 16 hr and treated with vehicle (DMSO) and serum-containing complete media (CM) alone or the indicated compounds (at 1, 10 or 20 μM) in CM. After 48 hr, the MTT assay was performed as described below. The visible absorbance of each well was quantified using a microplate reader.

FIG. 9 shows a synthetic scheme for the solid-phase synthesis of the compounds in soluble form of the present invention.

### DETAILED DESCRIPTION OF THE INVENTION

### I. GENERAL

The present invention provides novel compounds for the treatment of cancer. The compounds of the present invention are effective against cancer through the inhibition of cyclin kinase inhibitor p21.

p21 is an intracellular protein which functions in the regulatory cascades responsible for cell cycle progression and apoptosis. Without being bound by any theory, cyclin kinase inhibitors, such as p21, are thought to regulate cell cycle progression by binding to cyclin/cdk pairs and inhibiting their downstream activity on retinoblastoma (Rb) protein. Consequently the mitogenic transcription machinery is inhibited. Cyclin kinase inhibitors exert their anti-apoptotic effect by inhibiting the catalytic activities of kinases such as SAP and ASK1. Dysfunction of these regulatory cascades are hallmarks of cancer and other diseases.

Current chemotherapeutic agents for treating cancer can be divided into two classes: older generation agents that effect cell division or DNA synthesis; and newer generation agents which target specific molecular abnormalities in particular cancer types. Both classes have drawbacks. Older agents (e.g. cisplatin or nitrogen mustard) lack specificity; in addition to being cytotoxic to both normal and malignant cells. Newer agents, such as Imatinib (Gleevec), are effective only against a narrow spectrum of cancers. Compounds which target p21 offer to fill this gap in the available chemotherapeutic arsenal by providing specificity and efficacy against a range of cancer types.

Cancer treatments (chemotherapy and radiation) are designed to terminally damage the DNA of cancer cells and thereby induce their apoptosis. p21 inhibits apoptosis thus reducing the effectiveness of these treatment modalities. p21 inhibitors offer to enhance the efficacy of these treatments by counteracting p21's anti-apoptotic function.

p21 can be targeted by any variety of mechanisms, such as by interfering with its catalytic or binding activities. Alternatively, p21 protein levels can be regulated by altering gene transcription using anti-sense or siRNA techniques. Anti-sense and siRNA techniques act by reducing messenger RNA (mRNA) levels which reduces protein (p21) levels since mRNA is translated to produce the target protein, so less mRNA results in production of less target protein (p21).

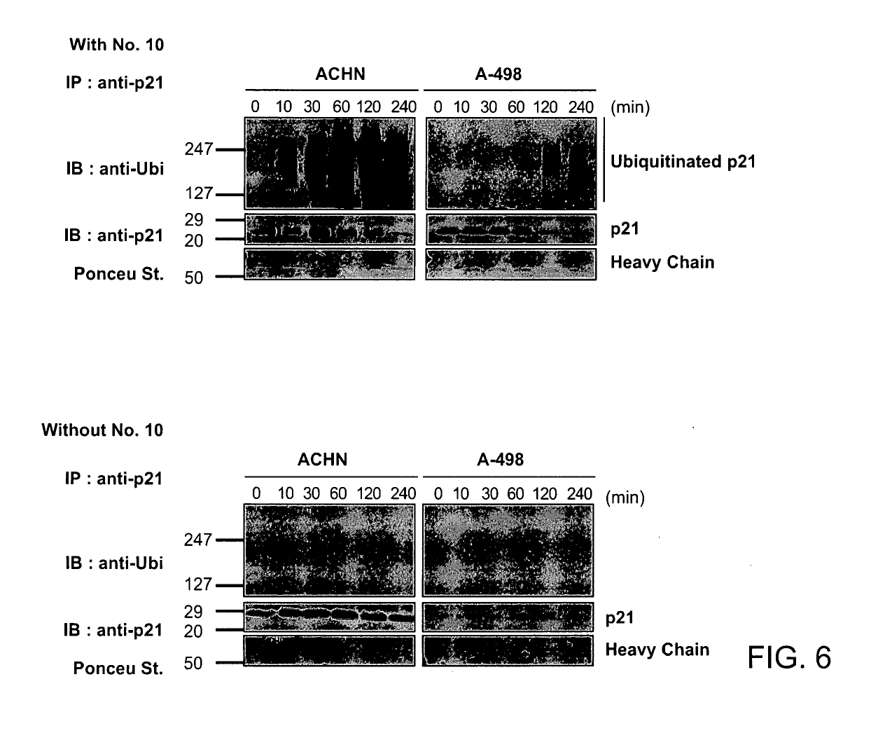

A compound of the present invention inhibits p21 activity, without limitation to any single theory or mode of action. FIG. 6 shows that in the presence of compound 10, p21 protein is bound by ubiquitin, which labels proteins for proteasomal destruction. Thus, without limitation to any single theory or mode of action, compound 10, in addition to other compounds of the present invention, reduces p21 activity by reducing the amount of p21 protein by inducing its ubiquitinylation and subsequent destruction.

A number of different signals identify target proteins for “ubiquitination.” One signal is the amino terminal residue of a protein. A protein with methionine at its N-terminus is less likely to be ubiquitinated than one with an arginine. Other signals that identify proteins for ubiquitination include cyclin destruction boxes, which are amino acid sequences that mark cell-cycle proteins for destruction and proteins rich in proline, glutamic acid, serine and threonine (PEST sequences). Denatured proteins, proteins with oxidized amino acids or those with hydrophobic patches on their surface, are also targets for ubiquitination.

Another signal identifying target proteins for ubiquitination is recognition of the ubiquitination signal occurs in a certain cellular compartment. That is, a protein is targeted for ubiquitination by its translocation from one cellular compartment to another, e.g., from the nucleus to the cytoplasm. This is shown to occur for cyclin D, p53 and p27 kip1.

Also, trans-targeting of ubiquitination is possible. In this scenario one protein, of a multi-protein complex, possesses a ubiquitination signal while another protein of the complex possesses the ubiquitin binding site. In this way the complex can be targeted. This is observed for the Cyclin/Cdk complex.

A link between cancer and p21 is observed. p21 over-expression is an early event in pancreatic neoplasms. High p21 levels are associated with poorer prognosis in breast cancer while p21 deficient cancers are more susceptible to chemotherapy. Several cancer types, such as oral, esophageal and breast have mutations in p21. Attenuating expression of p21 in colon cancer has salutary effects while modulating p21 sensitizes kidney cancer cells to chemotherapy.

Accordingly, without being bound by any theory, the compounds and compositions of the present invention cause dose-dependent cytotoxicity as well as apoptosis when exposed to cancer cells. In addition, the compounds and compositions work synergistically with other cancer treatment agents, such as doxorubicin, such that lower doses of the other cancer treatment agents are necessary. One of the ways in which the compounds of the present invention are effective at treating cancer is by specific induction of ubiquitin-dependent proteosome degradation of p21.

### II. DEFINITIONS

As used herein, the term “alkyl” refers to a straight or branched, saturated, aliphatic radical having the number of carbon atoms indicated. For example, C₁-C₆ alkyl includes, but is not limited to, methyl, ethyl, propyl, butyl, pentyl, hexyl, iso-propyl, iso-butyl, sec-butyl, tert-butyl, pentyl, hexyl, etc.

The term “lower” referred to above and hereinafter in connection with organic radicals or compounds respectively defines a compound or radical which can be branched or unbranched with up to and including 7, preferably up to and including 4 and (as unbranched) one or two carbon atoms.

As used herein, the term “alkenyl” refers to either a straight chain or branched hydrocarbon of 2 to 6 carbon atoms, having at least one double bond. Examples of alkenyl groups include, but are not limited to, vinyl, propenyl, isopropenyl, butenyl, isobutenyl, butadienyl, pentenyl or hexadienyl.

As used herein, the term “alkynyl” refers to either a straight chain or branched hydrocarbon of 2 to 6 carbon atoms, having at least one triple bond. Examples of alkynyl groups include, but are not limited to, acetylenyl, propynyl or butynyl.

As used herein, the term “halogen” refers to fluorine, chlorine, bromine and iodine.

As used herein, the term “haloalkyl” refers to alkyl as defined above where some or all of the hydrogen atoms are substituted with halogen atoms. For example, haloalkyl includes trifluoromethyl, flouromethyl, 1,2,3,4,5-pentafluoro-phenyl, etc. The term “perfluoro” defines a compound or radical which has at least two available hydrogens substituted with fluorine. For example, perfluorophenyl refers to 1,2,3,4,5-pentafluorophenyl, perfluoromethane refers to 1,1,1-trifluoromethyl, and perfluoromethoxy refers to 1,1,1-trifluoromethoxy.

As used herein, the term “alkoxy” refers to alkyl with the inclusion of an oxygen atom, for example, methoxy, ethoxy, etc. “Haloalkoxy” is as defined for alkoxy where some or all of the hydrogen atoms are substituted with halogen atoms. For example, halo-substituted-alkoxy includes trifluoromethoxy, etc.

As used herein, the term “cycloalkyl” refers to a saturated or partially unsaturated, monocyclic, fused bicyclic or bridged polycyclic ring assembly containing from 3 to 12 ring atoms, or the number of atoms indicated For example, C₃₋₈cycloalkyl includes cyclopropyl, cyclobutyl, cyclopentyl, cyclohexyl, and up to cyclooctyl.

As used herein, the term “heterocycloalkyl” refers to a ring system having from 3 ring members to about 20 ring members and from 1 to about 5 heteroatoms such as N, O and S. Additional heteroatoms can also be useful, including, but not limited to, B, Al, Si and P. The heteroatoms can also be oxidized, such as, but not limited to, —S(O)— and —S(O)₂—. For example, heterocycloalkyl includes, but is not limited to, tetrahydrofuranyl, tetrahydrothiophenyl, morpholino, pyrrolidinyl, pyrrolinyl, imidazolidinyl, imidazolinyl, pyrazolidinyl, pyrazolinyl, piperazinyl, piperidinyl, indolinyl, quinuclidinyl and 1,4-dioxa-8-aza-spiro[4.5]dec-8-yl.

As used herein, the term “aryl” refers to a monocyclic or fused bicyclic, tricyclic or greater, aromatic ring assembly containing 6 to 16 ring carbon atoms. For example, aryl can be phenyl, benzyl or naphthyl, preferably phenyl. “Arylene” means a divalent radical derived from an aryl group. Aryl groups can be mono-, di- or tri-substituted by one, two or three radicals selected from alkyl, alkoxy, aryl, hydroxy, halogen, cyano, amino, amino-alkyl, trifluoromethyl, alkylenedioxy and oxy-C₂-C₃-alkylene; all of which are optionally further substituted, for instance as hereinbefore defined; or 1- or 2-naphthyl; or 1- or 2-phenanthrenyl. Alkylenedioxy is a divalent substitute attached to two adjacent carbon atoms of phenyl, e.g. methylenedioxy or ethylenedioxy. Oxy-C₂-C₃-alkylene is also a divalent substituent attached to two adjacent carbon atoms of phenyl, e.g. oxyethylene or oxypropylene. An example for oxy-C₂-C₃-alkylene-phenyl is 2,3-dihydrobenzofuran-5-yl.

Preferred as aryl is naphthyl, phenyl or phenyl mono- or disubstituted by alkoxy, phenyl, halogen, alkyl or trifluoromethyl, especially phenyl or phenyl-mono- or disubstituted by alkoxy, halogen or trifluoromethyl, and in particular phenyl.

Examples of substituted phenyl groups as R are, e.g. 4-chlorophen-1-yl, 3,4-dichlorophen-1-yl, 4-methoxyphen-1-yl, 4-methylphen-1-yl, 4-aminomethylphen-1-yl, 4-methoxyethylaminomethylphen-1-yl, 4-hydroxyethylaminomethylphen-1-yl, 4-hydroxyethyl-(methyl)-aminomethylphen-1-yl, 3-aminomethylphen-1-yl, 4-N-acetylaminomethylphen-1-yl, 4-aminophen-1-yl, 3-aminophen-1-yl, 2-aminophen-1-yl, 4-phenyl-phen-1-yl, 4-(imidazol-1-yl)-phen-yl, 4-(imidazol-1-ylmethyl)-phen-1-yl, 4-(morpholin-1-yl)-phen-1-yl, 4-(morpholin-1-ylmethyl)-phen-1-yl, 4-(2-methoxyethylaminomethyl)-phen-1-yl and 4-(pyrrolidin-1-ylmethyl)-phen-1-yl, 4-(thiophenyl)-phen-1-yl, 4-(3-thiophenyl)-phen-1-yl, 4-(4-methylpiperazin-1-yl)-phen-1-yl, and 4-(piperidinyl)-phenyl and 4-(pyridinyl)-phenyl optionally substituted in the heterocyclic ring.

As used herein, the term “Heteroaryl” refers to a monocyclic or fused bicyclic or tricyclic aromatic ring assembly containing 5 to 16 ring atoms, where from 1 to 4 of the ring atoms are a heteroatom each N, O or S. For example, heteroaryl includes pyridyl, indolyl, indazolyl, quinoxalinyl, quinolinyl, isoquinolinyl, benzothienyl, benzofuranyl, furanyl, pyrrolyl, thiazolyl, benzothiazolyl, oxazolyl, isoxazolyl, triazolyl, tetrazolyl, pyrazolyl, imidazolyl, thienyl, or any other radicals substituted, especially mono- or di-substituted, by e.g. alkyl, nitro or halogen. Pyridyl represents 2-, 3- or 4-pyridyl, advantageously 2- or 3-pyridyl. Thienyl represents 2- or 3-thienyl. Quinolinyl represents preferably 2-, 3- or 4-quinolinyl. Isoquinolinyl represents preferably 1-, 3- or 4-isoquinolinyl. Benzopyranyl, benzothiopyranyl represents preferably 3-benzopyranyl or 3-benzothiopyranyl, respectively. Thiazolyl represents preferably 2- or 4-thiazolyl, and most preferred, 4-thiazolyl. Triazolyl is preferably 1-, 2- or 5-(1,2,4-triazolyl). Tetrazolyl is preferably 5-tetrazolyl.

Preferably, heteroaryl is pyridyl, indolyl, quinolinyl, pyrrolyl, thiazolyl, isoxazolyl, triazolyl, tetrazolyl, pyrazolyl, imidazolyl, thienyl, furanyl, benzothiazolyl, benzofuranyl, isoquinolinyl, benzothienyl, oxazolyl, indazolyl, or any of the radicals substituted, especially mono- or di-substituted.

Substituents for the aryl and heteroaryl groups are varied and are selected from: -halogen, —OR′, —OC(O)R′, —NR′R″, —SR′, —R′, —CN, —NO₂, —CO₂R′, —CONR′R″, —C(O)R′, —OC(O)NR′R″, —NR″C(O)R′, —NR″C(O)₂R′, —NR′—C(O)NR″R′″, —NH—C(NH₂)═NH, —NR′C(NH₂)═NH, —NH—C(NH₂)═NR′, —S(O)R′, —S(O)₂R′, —S(O)₂NR′R″, —N₃, —CH(Ph)₂, perfluoro(C₁-C₄)alkoxy, and perfluoro(C₁-C₄)alkyl, in a number ranging from zero to the total number of open valences on the aromatic ring system; and where R′, R″ and R′″ are independently selected from hydrogen, (C₁-C₈)alkyl and heteroalkyl, unsubstituted aryl and heteroaryl, (unsubstituted aryl)-(C₁-C₄)alkyl, and (unsubstituted aryl)oxy-(C₁-C₄)alkyl.

As used herein, the term “alkyl amine” refers to a straight or branched, saturated, radical having 1-10 carbon atoms and one or more amino groups. The alkyl portion of the alkyl amine is as defined above. The amino groups can be primary, secondary or tertiary. The alkyl amine can be further substituted with a hydroxy group. Alkyl amines useful in the present invention include, but are not limited to, ethyl amine, propyl amine, isopropyl amine, ethylene diamine and ethanolamine. One of skill in the art will appreciate that other alkyl amines are useful in the present invention.

As used herein, the term “alkyl-cycloalkyl” refers to a radical having an alkyl component and a cycloalkyl component, where the alkyl component links the cycloalkyl component to the compound. The alkyl component is as defined above, except that the alkyl component is at least divalent in order to link to the cycloalkyl component and to the compound. In some instances, the alkyl component can be absent. The cycloalkyl component is as defined above. Examples of alkyl-cycloalkyl include methylene-cyclohexane, among others.

As used herein, the term “alkyl-heterocycloalkyl” refers to a radical having an alkyl component and a heterocycloalkyl component, where the alkyl component links the heterocycloalkyl component to the compound. The alkyl component is as defined above, except that the alkyl component is at least divalent in order to link to the heterocycloalkyl component and to the compound. In some instances, the alkyl component can be absent. The heterocycloalkyl component is as defined above. Examples of alkyl-heterocycloalkyl include methylene-piperidinyl, among others.

As used herein, the term “alkyl-aryl” refers to a radical having an alkyl component and an aryl component, where the alkyl component links the aryl component to the compound. The alkyl component is as defined above, except that the alkyl component is at least divalent in order to link to the aryl component and to the compound. In some instances, the alkyl component can be absent. The aryl component is as defined above. Examples of alkyl-aryl include benzyl, among others.

As used herein, the term “alkyl-heteroaryl” refers to a radical having an alkyl component and a heteroaryl component, where the alkyl component links the heteroaryl component to the compound. The alkyl component is as defined above, except that the alkyl component is at least divalent in order to link to the heteroaryl component and to the compound. In some instances, the alkyl component can be absent. The heteroaryl component is as defined above. Examples of alkyl-heteroaryl include methylene-pyridyl, among others.

As used herein, the term “alkenyl-aryl” refers to a radical having both an alkenyl component and a aryl component, where the alkenyl component links the aryl component to the compound. The alkenyl component is as defined above, except that the alkenyl component is at least divalent in order to link to the aryl component and to the compound. The aryl component is as defined above. Examples of alkenyl-aryl include ethenyl-phenyl, among others.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine.

“Amino acid analogs” refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid.

“Unnatural amino acids” are not encoded by the genetic code and can, but do not necessarily have the same basic structure as a naturally occurring amino acid. Unnatural amino acids include, but are not limited to azetidinecarboxylic acid, 2-aminoadipic acid, 3-aminoadipic acid, beta-alanine, aminopropionic acid, 2-aminobutyric acid, 4-aminobutyric acid, 6-aminocaproic acid, 2-aminoheptanoic acid, 2-aminoisobutyric acid, 3-aminoisbutyric acid, 2-aminopimelic acid, tertiary-butylglycine, 2,4-diaminoisobutyric acid, desmosine, 2,2′-diaminopimelic acid, 2,3-diaminopropionic acid, N-ethylglycine, N-ethylasparagine, homoproline, hydroxylysine, allo-hydroxylysine, 3-hydroxyproline, 4-hydroxyproline, isodesmosine, allo-isoleucine, N-methylalanine, N-methylglycine, N-methylisoleucine, N-methylpentylglycine, N-methylvaline, naphthalanine, norvaline, ornithine, pentylglycine, pipecolic acid and thioproline.

“Amino acid mimetics” refers to chemical compounds that have a structure that is different from the general chemical structure of an amino acid, but that functions in a manner similar to a naturally occurring amino acid.

Amino acids can be referred to herein by either the commonly known three letter symbols or by the one-letter symbols recommended by the IUPAC-IUB Biochemical Nomenclature Commission. Nucleotides, likewise, can be referred to by their commonly accepted single-letter codes.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
(see, e.g., Creighton, Proteins (1984)).

As used herein, the term “salt” refers to acid or base salts of the compounds used in the methods of the present invention. Illustrative examples of pharmaceutically acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

Pharmaceutically acceptable salts of the acidic compounds of the present invention are salts formed with bases, namely cationic salts such as alkali and alkaline earth metal salts, such as sodium, lithium, potassium, calcium, magnesium, as well as ammonium salts, such as ammonium, trimethyl-ammonium, diethylammonium, and tris-(hydroxymethyl)-methyl-ammonium salts.

Similarly acid addition salts, such as of mineral acids, organic carboxylic and organic sulfonic acids, e.g., hydrochloric acid, methanesulfonic acid, maleic acid, are also possible provided a basic group, such as pyridyl, constitutes part of the structure.

The neutral forms of the compounds can be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

As used herein, the term “hydrate” refers to a compound that is complexed to at least one water molecule. The compounds of the present invention can be complexed with from 1 to 10 water molecules.

As used herein, the term “prodrug” refers to covalently bonded carriers which are capable of releasing the active agent of the methods of the present invention, when the prodrug is administered to a mammalian subject. Release of the active ingredient occurs in vivo. Prodrugs can be prepared by techniques known to one skilled in the art. These techniques generally modify appropriate functional groups in a given compound. These modified functional groups however regenerate original functional groups by routine manipulation or in vivo. Prodrugs of the active agents of the present invention include active agents wherein a hydroxy, amidino, guanidino, amino, carboxylic, carbamate, urea or a similar group is modified.

As used herein, the term “pharmaceutically acceptable excipient” refers to a substance that aids the administration of an active agent to and/or absorption by a subject. Pharmaceutically acceptable excipients useful in the present invention include, but are not limited to, binders, fillers, disintegrants, lubricants, coatings, sweeteners, flavors and colors. One of skill in the art will recognize that other pharmaceutical excipients are useful in the present invention.

“Inhibition”, “inhibits” and “inhibitor” refer to a compound that prohibits or a method of prohibiting, a specific action or function.

As used herein, “administering” refers to oral administration, administration as a suppository, topical contact, parenteral, intravenous, intraperitoneal, intramuscular, intralesional, intranasal or subcutaneous administration, intrathecal administration, or the implantation of a slow-release device e.g., a mini-osmotic pump, to the subject.

“Subject” refers to animals such as mammals, including, but not limited to, primates (e.g., humans), cows, sheep, goats, horses, dogs, cats, rabbits, rats, mice and the like. In certain embodiments, the subject is a human.

As used herein, the terms “therapeutically effective amount” refers to a dose that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). In sensitized cells, the therapeutically effective dose can often be lower than the conventional therapeutically effective dose for non-sensitized cells.

As used herein, the terms “treat”, “treating” and “treatment” refers to any indicia of success in the treatment or amelioration of an injury, pathology, condition, or symptom (e.g., pain), including any objective or subjective parameter such as abatement; remission; diminishing of symptoms or making the symptom, injury, pathology or condition more tolerable to the patient; decreasing the frequency or duration of the symptom or condition; or, in some situations, preventing the onset of the symptom or condition. The treatment or amelioration of symptoms can be based on any objective or subjective parameter; including, e.g., the result of a physical examination.

As used herein, the term “cancer” refers to the presence of cells possessing characteristics typical of cancer-causing cells, such as uncontrolled proliferation, immortality, metastatic potential, rapid growth and proliferation rate, and certain characteristic morphological features. Often, cancer cells will be in the form of a tumor, but such cells can exist alone within an animal, or can circulate in the blood stream as independent cells, such as leukemic cells.

### III. COMPOUNDS

The compounds of the present invention include those compounds that inhibit cyclin kinase inhibitor p21. In some embodiments, the compounds of the present invention can be a compound of formula I:

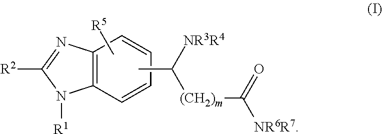

In Formula I, radical R¹ is C₁₋₆ alkyl, C₀₋₆ alkyl-NR⁸R⁹, C₀₋₆ alkyl-cycloalkyl, C₀₋₆ alkyl-heterocycloalkyl, C₀₋₆ alkyl-aryl and C₀₋₆ alkyl-heteroaryl, each optionally substituted with from 1-4 R^{1a} groups. Within R¹, each radical R^{1a} is independently H, halogen, C₁₋₆ alkyl, C₁₋₆ haloalkyl, C₂₋₆ alkenyl, C₂₋₆ alkynyl, C₁₋₆ alkoxy, C₁₋₆ haloalkoxy, C₁₋₆ hydroxyalkyl, —OH, —C₀₋₆ alkyl-NR⁸R⁹, —SR⁸, —C(O)R⁸, —C₀₋₆ alkyl-C(O)OR⁸, —C(O)NR⁸R⁹, —N(R⁸)C(O)R⁹, —N(R⁸)C(O)OR⁹, —N(R⁸)C(O)NR⁸R⁹, —OP(O)(OR⁸)₂, —S(O)₂OR⁸, —S(O)₂NR⁸R⁹, —CN, C₀₋₆ alkyl-cycloalkyl, heterocycloalkyl, C₀₋₆ alkyl-aryl and heteroaryl, alternatively, two R^{1a} groups are joined to form ═O. In Formula I, radical R² is C₀₋₆ alkyl-aryl, C₂₋₆ alkenyl-aryl, C₀₋₆ alkyl-heteroaryl, and —N(R⁸)-aryl, each optionally substituted with 1-4 members that are each independently R^{1a}, —OR¹⁰, —SR¹⁰ or —NR⁸R¹⁰. Radical R³ of Formula I is —C(O)—C₁₋₆ alkyl, —C(O)—C₁₋₆ hydroxyalkyl, —C(O)—C₁₋₆ alkylamine —C(O)-heterocycloalkyl, —C(O)—NR^{3a}R^{3b}, —C(O)OR^{3a}, —S(O)₂R^{3a} or an amino acid. Within R³, each of radicals R^{3a} and R^{3b} are independently H, C₁₋₆ alkyl, cycloalkyl, heterocycloalkyl, aryl, or heteroaryl, each optionally substituted with from 1-4 R⁸ groups. Radicals R⁴, R⁵, R⁶ and R⁷ of Formula I, as well as radical R⁸ of R^{1a}, R^{3a} and R^{3b}, and radical R⁹ of R^{1a}, are each independently H or C₁₋₆ alkyl. Radical R¹⁰ of Formula I is cycloalkyl, heterocycloalkyl, aryl or heteroaryl, each optionally substituted with from 1-4 R^{1a} groups. Subscript m of Formula I is an integer from 0-2. The present invention also includes the salts, hydrates, prodrugs, and isomers of the compounds of Formula I.

In some other embodiments, radical R¹ of Formula I is C₁₋₆ alkyl, C₀₋₆ alkyl-cycloalkyl, C₀₋₆ alkyl-heterocycloalkyl, C₀₋₆ alkyl-aryl or C₀₋₆ alkyl-heteroaryl, each optionally substituted with from 1-4 R^{1a} groups. Within R¹, each radical R^{1a} is independently H, halogen, C₁₋₆ alkyl, C₁₋₆ haloalkyl, C₂₋₆ alkenyl, C₂₋₆ alkynyl, C₁₋₆ alkoxy, C₁₋₆ haloalkoxy, C₁₋₆ hydroxyalkyl, —OH, —C₀₋₆ alkyl-NR⁸R⁹, —SR⁸, —C(O)R⁸, —C₀₋₆ alkyl-C(O)OR⁸, —C(O)NR⁸R⁹, —N(R⁸)C(O)R⁹, —N(R⁸)C(O)OR⁹, —N(R⁸)C(O)NR⁸R⁹, —OP(O)(OR⁸)₂, —S(O)₂OR⁸, —S(O)₂NR⁸R⁹, —CN, C₀₋₆ alkyl-cycloalkyl, heterocycloalkyl, C₀₋₆ alkyl-aryl or heteroaryl. In Formula I, radical R² is C₀₋₆ alkyl-aryl, C₂₋₆ alkenyl-aryl, or C₀₋₆ alkyl-heteroaryl, each optionally substituted with from 1-4 R^{1a} groups. Radical R³ of Formula I is —C(O)—C₁₋₆ alkyl, —C(O)—C₁₋₆ hydroxyalkyl, —C(O)—C₁₋₆ alkylamine, —C(O)-heterocycloalkyl, —C(O)—NR^{3a}R^{3b}, —C(O)OR^{3a}, —S(O)₂R^{3a} or an amino acid. Within R³, each of radicals R^{3a} and R^{3b} are independently H, C₁₋₆ alkyl, cycloalkyl, heterocycloalkyl, aryl, or heteroaryl, each optionally substituted with from 1-4 R⁸ groups. Radicals R⁴, R⁵, R⁶ and R⁷ of Formula I, as well as radical R⁸ of R^{1a}, R^{3a} and R^{3b}, and radical R⁹ of R^{1a}, are each independently H or C₁₋₆ alkyl. And subscript m of Formula I is an integer from 0-2.

In other embodiments, the compound is of formula Ia:

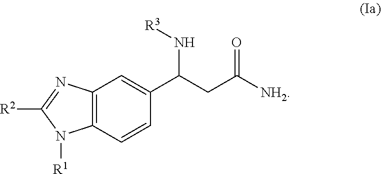

In some other embodiments, R¹ is C₀₋₆ alkyl-cycloalkyl, or C₀₋₆ alkyl-heterocycloalkyl, each optionally substituted with H, C₁₋₆ alkyl, or C₀₋₆ alkyl-aryl. R² is aryl, C₂₋₆ alkenyl-aryl, or heteroaryl, each optionally substituted with H, halogen, C₁₋₆ alkyl, C₁₋₆ alkoxy, or —OH. And R³ is —C(O)—C₁₋₆ alkyl, —C(O)—C₁₋₆ alkylamine, —C(O)—NR^{3a}R^{3b}, or an amino acid. In still other embodiments, R¹ is C₀₋₆ alkyl-piperidinyl. R² is phenyl or anthracenyl. And R³ is —C(O)—NHR^{3a}, wherein R^{3a} is phenyl, optionally substituted with C₁₋₆ alkyl.

In some embodiments, R¹ is

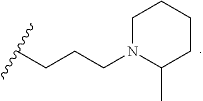

In other embodiments, R² is

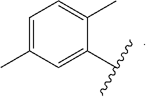

In some other embodiments, R³ is

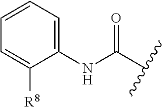

wherein R⁸ is C₁₋₆ alkyl.

In still other embodiments, the compound can be

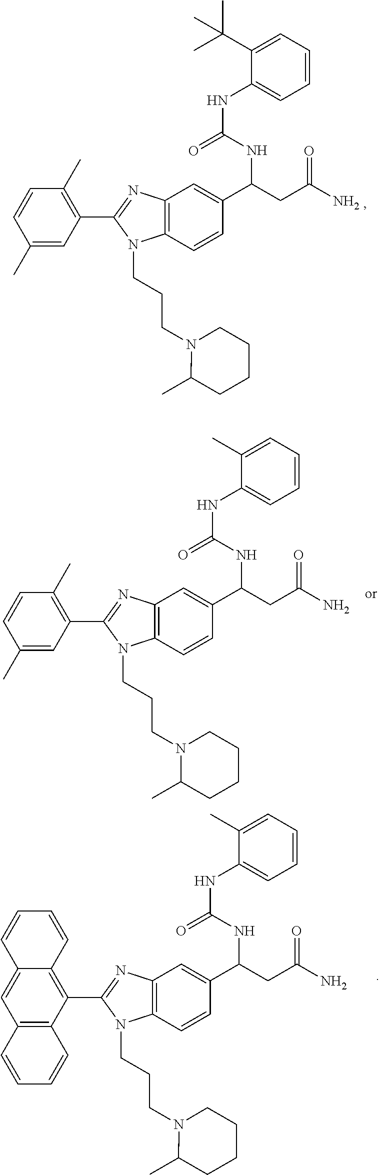

In other embodiments, R¹ is C₀₋₆ alkyl-NR⁸R⁹, C₀₋₆ alkyl-cycloalkyl, or C₀₋₆ alkyl-heterocycloalkyl, each optionally substituted with from 1-4 R^{1a} groups. R² is aryl, C₂₋₆ alkenyl-aryl, heteroaryl, aryl-O-aryl, or —NH-aryl, each optionally substituted with from 1-4 R^{1a} groups. And R³ is —C(O)—C₁₋₆ alkyl, —C(O)—C₁₋₆ alkylamine, —C(O)—NR^{3a}R^{3b}, or an amino acid.

In some other embodiments, R¹ is C₀₋₆ alkyl-piperidinyl or C₀₋₆ alkyl-NR⁸R⁹, each optionally substituted with from 1-4 R^{1a} groups. R² is phenyl-O-phenyl, optionally substituted with from 1-4 R^{1a} groups. And R³ is —C(O)—NHR^{3a}, wherein R^{3a} is phenyl, optionally substituted with C₁₋₆ alkyl.

In some other embodiments, R² is

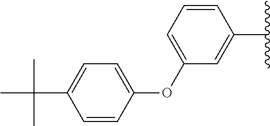

In still other embodiments, the compound is of formula Ib:

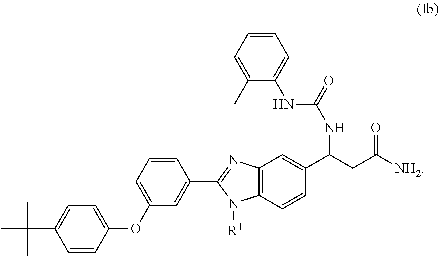

In yet other embodiments, the compound is:

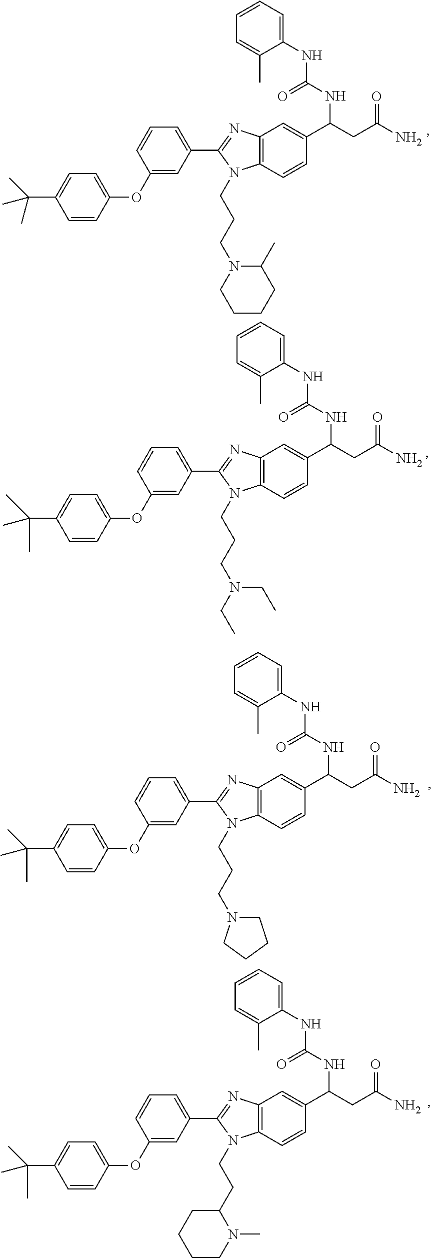

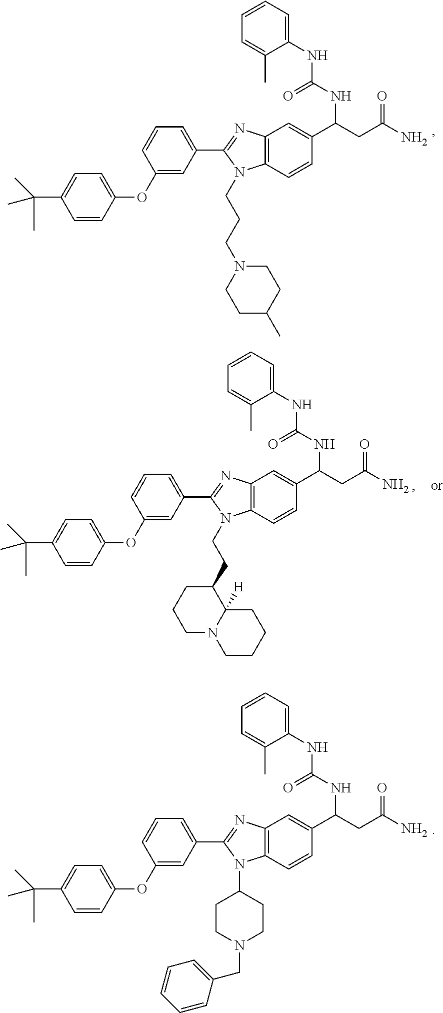

Certain compounds of the present invention possess asymmetric carbon atoms (optical centers) or double bonds; the racemates, diastereomers, geometric isomers and individual isomers are all intended to be encompassed within the scope of the present invention.

The compounds of the present invention also include the prodrug and metabolite forms of the compounds of Formula I. In addition, the compounds of Formula I include the salts, hydrates and solvates of these compounds.

The library compounds on beads of the present invention can be prepared by a variety of methods known to one of skill in the art, including using an encoded small molecule “one-bead one-compound” (OBOC) combinatorial library (U.S. application Ser. No. 10/811,331, filed Mar. 25, 2004, incorporated herein in its entirety).

The OBOC method involves a MS-encoded small molecule OBOC combinatorial library synthesized on topological bilayer beads such that the testing molecule is displayed on the bead surface (to interact with the target protein), and the coding tags are located in the interior of the beads (with no access to the target protein). Thus, interference of coding tags in the screening process was excluded. The coding tags are decoded using MALDI-TOF MS (see Methods).

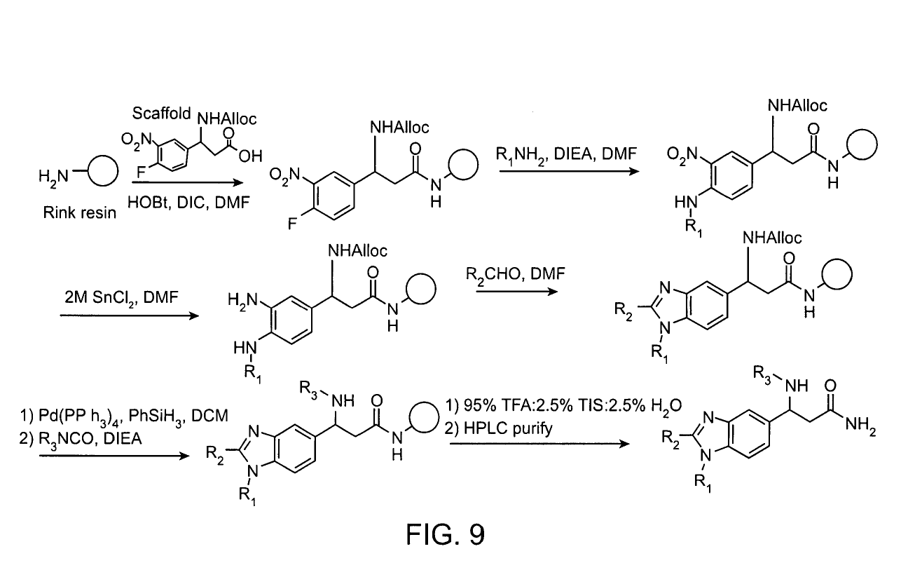

The compounds can be prepared by a variety of methods known to one of skill in the art. For example, using the synthetic scheme in FIG. 9, the compounds were prepared and identified as inhibitors of cyclin kinase inhibitor p21. Additional methods of making the compounds of the present invention include solid-phase and solution-phase synthetic methods are known to one of skill in the art.

TABLE 1
Inhibitors of cyclin kinase inhibitor p21
(Ia)

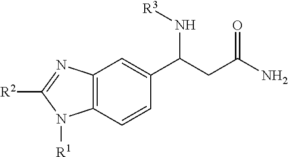

| | | | | | MAL- |
|---|---|---|---|---|---|
| | | | | | DI- |
| | | | | | TOF |
| | | | | | MS |
| | | | | Calc'd | M.W. |
| Entry | R₁ | R₂ | R₃ | M.W. | (MH⁺) |
| No. 1 | 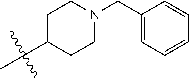 | 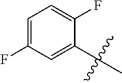 | 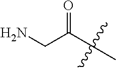 | | |
| No. 2 | 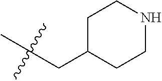 | 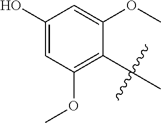 | 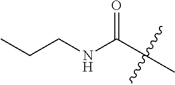 | | |
| No. 3 | 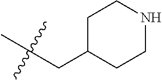 | 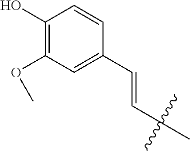 | 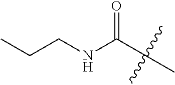 | | |
| No. 4 | 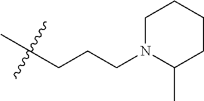 | 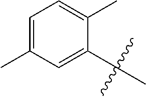 | 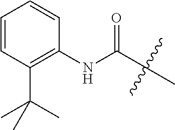 | 622.84 | 622.40 |
| No. 5 | 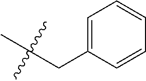 | 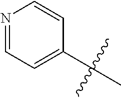 | 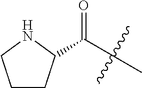 | | |
| No. 6 | 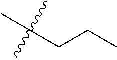 | 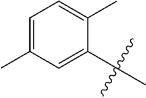 | 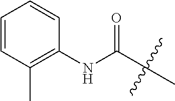 | | |
| No. 7 | 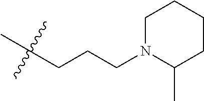 | 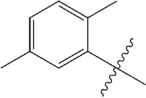 | 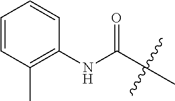 | 580.35 | 581.34 |
| No. 8 | 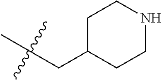 | 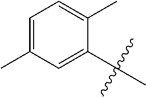 | 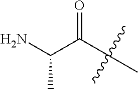 | | |
| No. 9 | 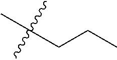 | 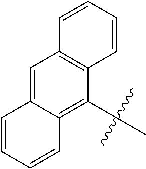 | 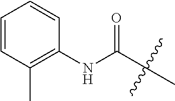 | | |
| No. 10 | 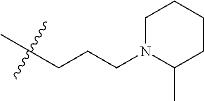 | 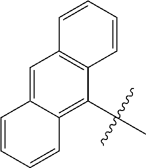 | 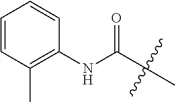 | 652.35 | 653.34 |
| No. 11 | 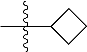 | 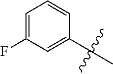 | 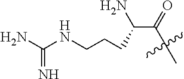 | | |
| No. 12 | 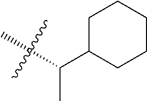 | 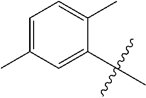 | 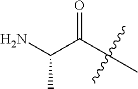 | | |
| LLW1 | 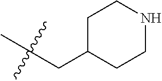 | 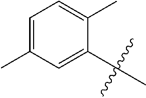 | 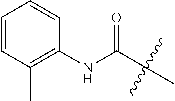 | 538.31 | 539.28 |
| LLW2 | 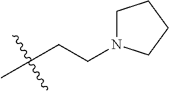 | 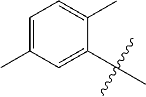 | 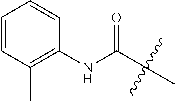 | 538.31 | 539.28 |
| LLW3 | 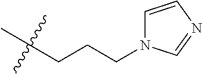 | 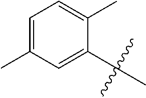 | 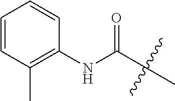 | 549.29 | 550.26 |
| LLW4 | 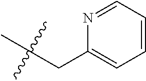 | 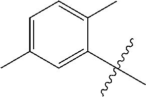 | 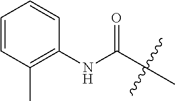 | 532.26 | 533.22 |
| LLW5 | 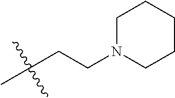 | 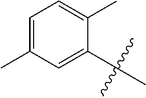 | 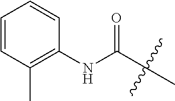 | 552.32 | 533.28 |
| LLW6 | 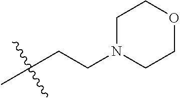 | 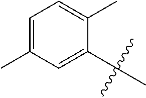 | 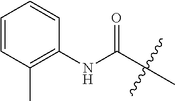 | 554.30 | 555.28 |
| LLW7 | 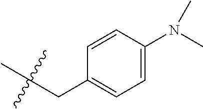 | 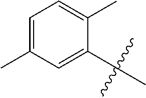 | 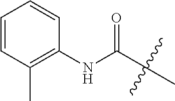 | 574.31 | 575.28 |
| LLW8 | 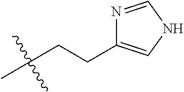 | 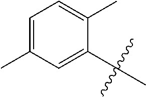 | 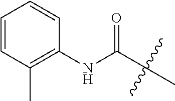 | 535.27 | 536.25 |
| LLW11 | 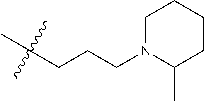 | 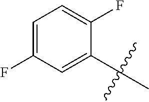 | 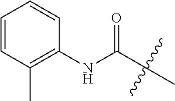 | 588.30 | 589.29 |
| LLW12 | 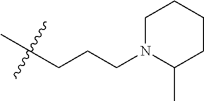 | 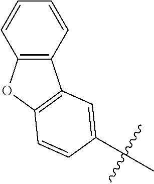 | 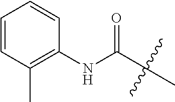 | 642.33 | 643.32 |
| LLW13 | 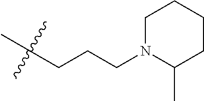 | 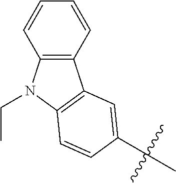 | 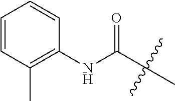 | 669.38 | 670.35 |
| LLW14 | 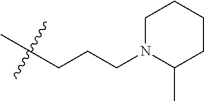 | 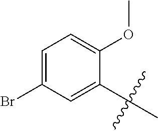 | 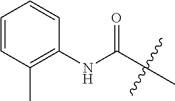 | 660.24, 662.24 | 661.22, 663.22 |
| LLW15 | 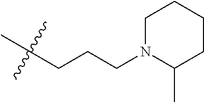 | 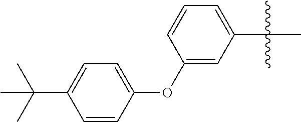 | 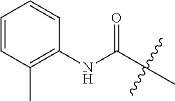 | 700.41 | 701.38 |
| LLW16 | 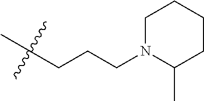 | 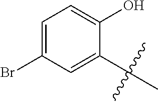 | 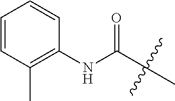 | 646.23, 648.23 | 647.20, 649.20 |
| LLW17 | 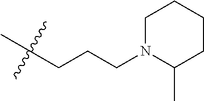 | 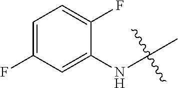 | 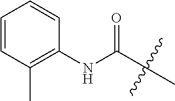 | 603.31 | 604.28 |
| LLW18 | 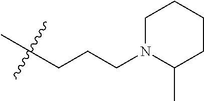 | 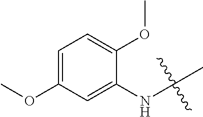 | 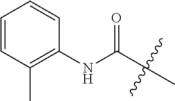 | 627.35 | 628.32 |
| LLW33 | 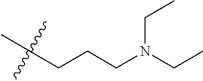 | 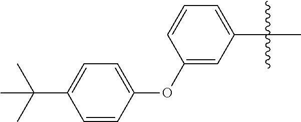 | 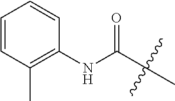 | 674.39 | 675.42 |
| LLW34 | 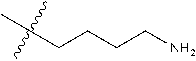 | 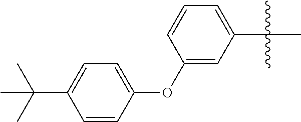 | 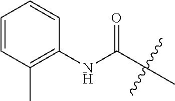 | 632.35 | 633.39 |
| LLW35 | 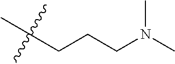 | 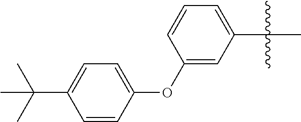 | 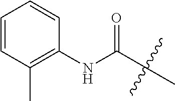 | 646.36 | 647.38 |
| LLW36 | 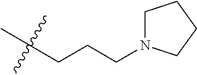 | 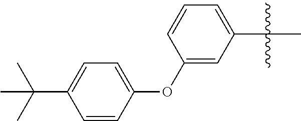 | 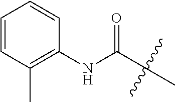 | 672.38 | 673.38 |
| LLW37 | 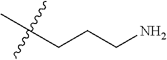 | 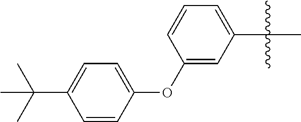 | 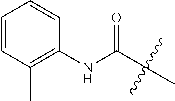 | 618.33 | 619.37 |
| LLW38 | 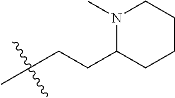 | 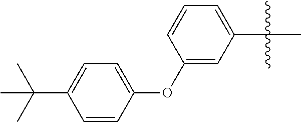 | 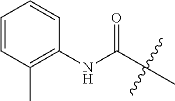 | 686.39 | 687.39 |
| LLW39 | 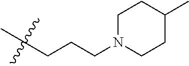 | 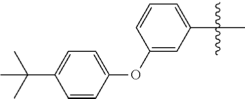 | 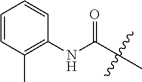 | 700.41 | 701.37 |
| LLW40 | 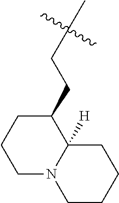 | 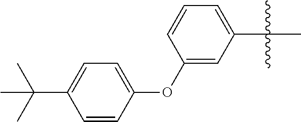 | 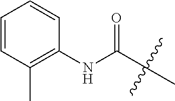 | 726.43 | 727.41 |
| LLW41 | 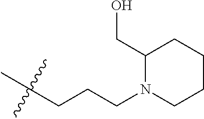 | 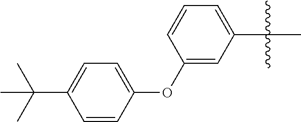 | 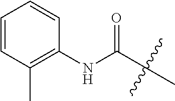 | 716.41 | 717.39 |
| LLW42 | 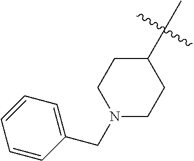 | 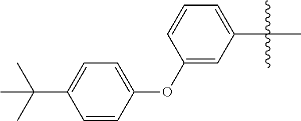 | 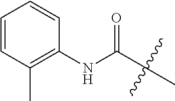 | 734.39 | 735.32 |
| LLW43 | 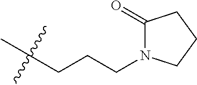 | 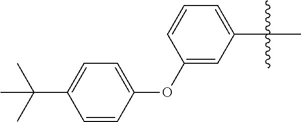 | 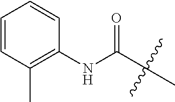 | 686.36 | 687.32 |
| LLW44 | 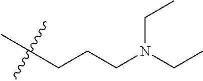 | 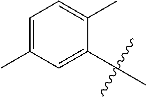 | 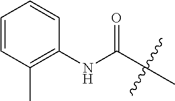 | 554.34 | 555.32 |
| LLW45 | 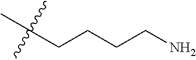 | 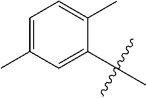 | 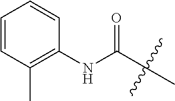 | 512.29 | 513.37 |
| LLW46 | 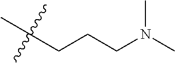 | 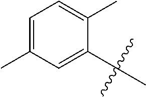 | 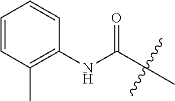 | 526.31 | 727.31 |
| LLW47 | 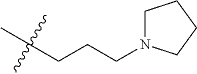 | 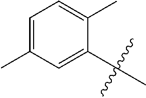 | 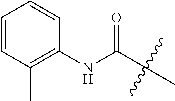 | 552.32 | 553.33 |
| LLW48 | 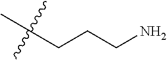 | 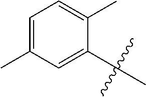 | 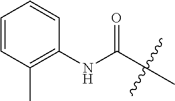 | 498.27 | 499.27 |
| LLW49 | 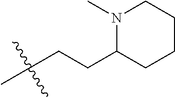 | 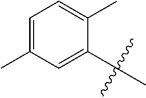 | 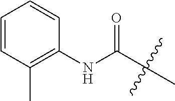 | 566.34 | 567.31 |
| LLW50 | 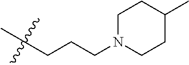 | 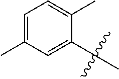 | 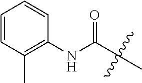 | 580.35 | 581.35 |
| LLW51 | 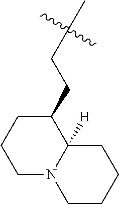 | 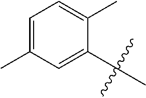 | 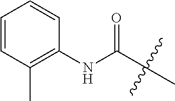 | 606.37 | 607.36 |
| LLW52 | 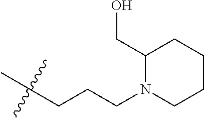 | 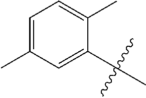 | 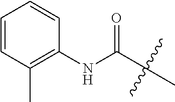 | 596.35 | 597.32 |
| LLW53 | 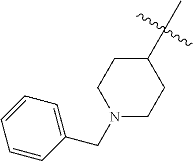 | 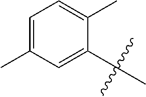 | 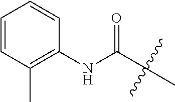 | 614.34 | 615.33 |
| LLW54 | 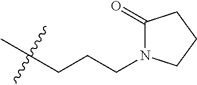 | 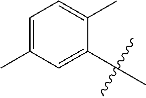 | 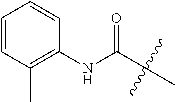 | 566.30 | 567.35 |
| LLW55 | 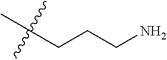 | 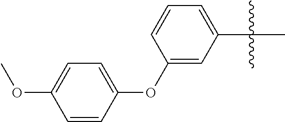 | 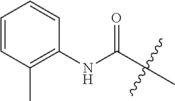 | 592.28 | 593.22 |

### IV. COMPOSITIONS

The compounds of the present invention can be formulated in a variety of different manners known to one of skill in the art. Pharmaceutically acceptable carriers are determined in part by the particular composition being administered, as well as by the particular method used to administer the composition. Accordingly, there are a wide variety of suitable formulations of pharmaceutical compositions of the present invention (see, e.g., Remington's Pharmaceutical Sciences, 20^{th} ed., 2003, supra). Effective formulations include oral and nasal formulations, formulations for parenteral administration, and compositions formulated for extended release.

In some embodiments, the present invention provides a pharmaceutical composition including a compound of the present invention and a pharmaceutically acceptable excipient.

A. Formulations

Formulations suitable for oral administration can consist of (a) liquid solutions, such as an effective amount of a compound of the present invention suspended in diluents, such as water, saline or PEG 400; (b) capsules, sachets, depots or tablets, each containing a predetermined amount of the active ingredient, as liquids, solids, granules or gelatin; (c) suspensions in an appropriate liquid; (d) suitable emulsions; and (e) patches. The pharmaceutical forms can include one or more pharmaceutically acceptable excipients includine lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The composition can, if desired, also contain other compatible therapeutic agents. Preferred pharmaceutical preparations can deliver the compounds of the invention in a sustained release formulation.

Pharmaceutical preparations useful in the present invention also include extended-release formulations. In some embodiments, extended-release formulations useful in the present invention are described in U.S. Pat. No. 6,699,508, which can be prepared according to U.S. Pat. No. 7,125,567, both patents incorporated herein by reference.

B. Administration

The pharmaceutical preparations are typically delivered to a mammal, including humans and non-human mammals. Non-human mammals treated using the present methods include domesticated animals (i.e., canine, feline, murine, rodentia, and lagomorpha) and agricultural animals (bovine, equine, ovine, porcine).

The pharmaceutical compositions can be administered to the patient in a variety of ways, including topically, parenterally, intravenously, intradermally, subcutaneously, intramuscularly, colonically, rectally or intraperitoneally. Preferably, the pharmaceutical compositions are administered parenterally, topically, intravenously, intramuscularly, subcutaneously, orally, or nasally, such as via inhalation.

In practicing the methods of the present invention, the pharmaceutical compositions can be used alone, or in combination with other therapeutic or diagnostic agents.

The compounds of the present invention can be administered as frequently as necessary, including hourly, daily, weekly or monthly. The compounds utilized in the pharmaceutical method of the invention are administered at the initial dosage of about 0.0001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, can be varied depending upon the requirements of the patient, the severity of the condition being treated, and the compound being employed. For example, dosages can be empirically determined considering the type and stage of disease diagnosed in a particular patient. The dose administered to a patient, in the context of the present invention should be sufficient to effect a beneficial therapeutic response in the patient over time. The size of the dose also will be determined by the existence, nature, and extent of any adverse side-effects that accompany the administration of a particular compound in a particular patient. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage can be divided and administered in portions during the day, if desired. Doses can be given daily, or on alternate days, as determined by the treating physician. Doses can also be given on a regular or continuous basis over longer periods of time (weeks, months or years), such as through the use of a subdermal capsule, sachet or depot, or via a patch.

In practicing the methods of the present invention, the pharmaceutical compositions can be used alone, or in combination with other therapeutic or diagnostic agents. The additional drugs used in the combination protocols of the present invention can be administered separately or one or more of the drugs used in the combination protocols can be administered together, such as in an admixture. Where one or more drugs are administered separately, the timing and schedule of administration of each drug can vary. The other therapeutic or diagnostic agents can be administered at the same time as the compounds of the present invention, separately or at different times.

### V. ASSAY TO IDENTIFY COMPOUNDS FOR BINDING TO p21

The compounds of the present invention can be identified as inhibitors of cyclin kinase inhibitor p21 by a variety of assays and methods known to one of skill in the art. In some embodiments, inhibitors of cyclin kinase inhibitor p21 are identified using the assay described in Example 3.

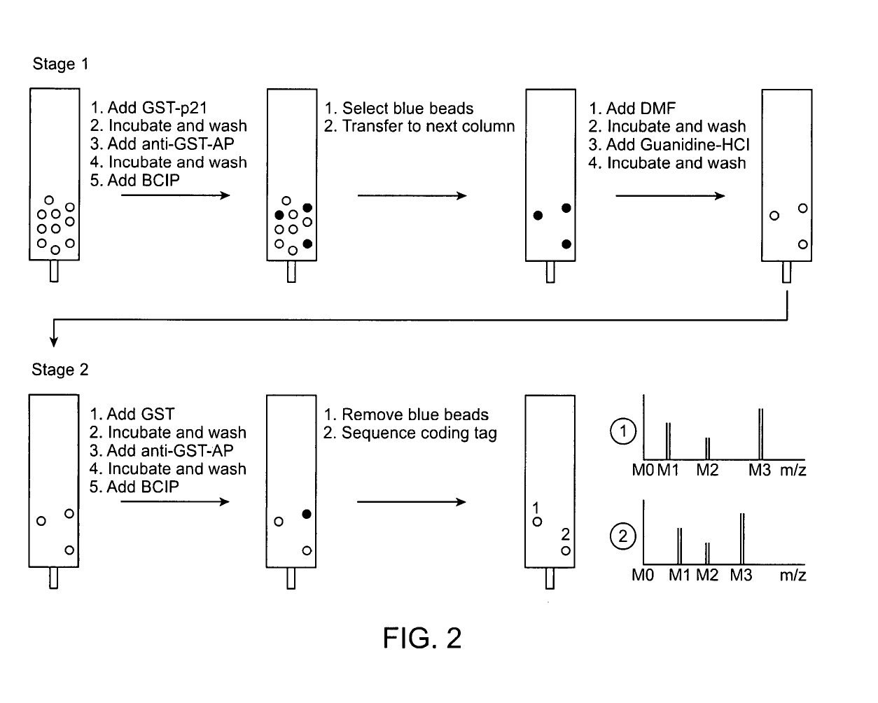

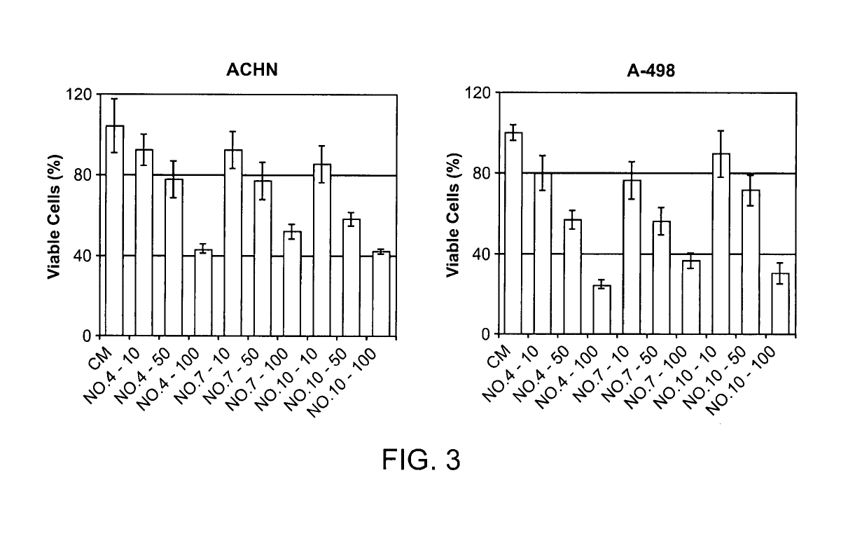

Other assays for identifying inhibitors of cyclin kinase inhibitor p21 involve screening the encoded OBOC small molecule library screened with an enzyme-linked two step subtraction colorimetric assay using recombinant GST-tagged p21 as the screening probe (FIG. 2). The compound-beads can be isolated and decoded, thereby identifying the active compounds. Each of these candidate small molecules can then be resynthesized and tested individually. Following this assay method, at least 3 of the compounds of the present invention (Nos. 4, 7 and 10) were found to be biologically active in a cell-based MTT cytotoxic assay (FIG. 3), each showing dose-dependent inhibition of cell survival from 10-100 μM.

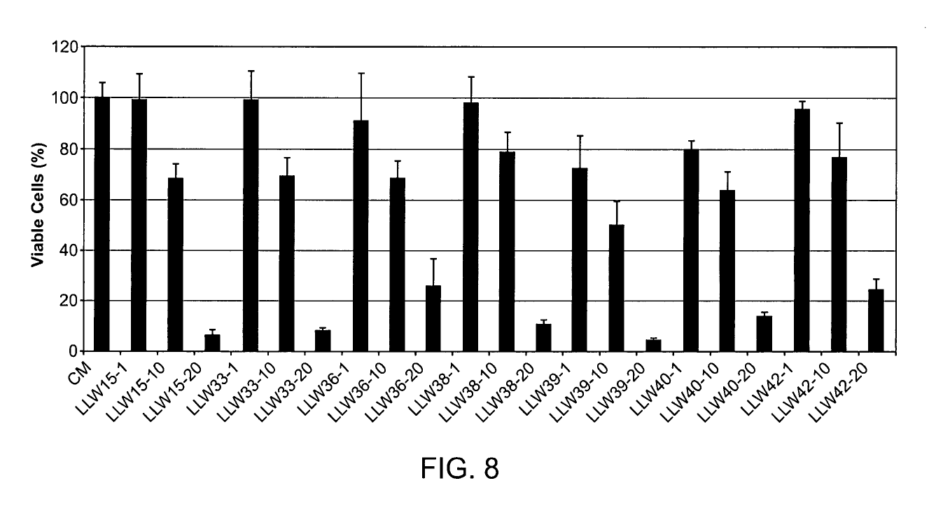

This assay method also identified compounds LLW15, LLW33, LLW36, LLW38, LLW39, LLW40 and LLW42 of the present invention as biologically active in a cell-based MTT cytotoxic assay (FIG. 8), each showing dose-dependent inhibition of cell survival from 1-20 μM.

### VI. METHODS OF INHIBITING CYCLIN KINASE INHIBITOR p21

The compounds of the present invention are useful for the treatment of a variety of cancers, as well as the inhibition of cyclin kinase inhibitor p21. Over-expression or increased cytoplasmic p21 is associated with a variety of disease conditions, such as cancer. Accordingly, inhibitors of p21, such as the compounds of the present invention, are useful for the treatment of a variety of disease states, such as cancer.

Cancers that can be treated by the compounds and compositions of the present invention are members of larger classes of cancer types which are defined by histological appearance. For example, cancers of the mouth, colon, esophagus and breast are often carcinomas (squamous cell or adeno-). Other classes of cancer include embryonal (blastoma), lymphoma and sarcomas (mesenchymal).

In some embodiments, the present invention provides a method of inhibiting cyclin kinase inhibitor p21 including administering to a subject in need thereof a therapeutically effective amount of a compound of the present invention.

In other embodiments, the present invention provides a method of treating cancer, the method including administering to a subject in need thereof a therapeutically effective amount of a compound of the present invention. The cancer can be any cancer known to one of skill in the art, such as, kidney and breast cancer. In some other embodiments, the cancer is kidney cancer. In still other embodiments, the cancer is breast cancer. In still yet other embodiments, the cancer is pancreatic cancer.

In some other embodiments, compounds of the present invention can be combined with other compound and compositions for the treatment of cancer or the inhibition of cyclin kinase inhibitor p21. Other compounds and compositions that can be combined with the compounds and compositions of the present invention are known to treat cancer or inhibit cyclin kinase inhibitor p21 and include, but are not limited to, doxorubicin, cisplatinum, cyclophosphamide, chlorambucil and nitrogen mustard. In some embodiments, the other compound is doxorubicin.

The components of the combination can be administered together or separately. The components of the combination can be administered simultaneously, during the same hour, day, week or month, or during the same therapy. The components of the combination or the combination thereof can be administered periodically, e.g. hourly, daily, weekly or biweekly, or monthly, depending on the patient's needs. Alternatively, the components of the combination or the combination can be administered several times a day, several times a week, several times a month or several times a year.

### VII. EXAMPLES

Rink amide MBHA resin (0.59 mmol/g) was purchased from Tianjin Nankai Hecheng (Tianjin, China). TentaGel S NH₂ resin (TG resin) was purchased from Rapp Polymere Gmbh (Tubingen, Germany). N-Hydroxybenzotriazole (HOBt) and 1,3-diisopropylcarbodiimide (DIC) were purchased from Advanced ChemTech (Louisville, Ky.). Dichloromethane (DCM), methanol (MeOH), diethyl ether and acetonitrile (CH₃CN) were purchased from Fisher (Houston, Tex.). N,N-dimethylformamide (DMF) was purchased from VWR (Brisbane, Calif.). Mouse anti-β-actin monoclonal antibody and the following chemicals and solvents (cycloheximide, dimethyl sulfoxide (DMSO), glycerol, glycine, glycerophosphate, lactacystin, LLnL, MG-132, sodium vanadate, sodium chloride, Thiazolyl Blue Tetrazolium Bromide, Trizma base, Tween 20, Cisplatin and Doxorubicin) were from Sigma (St. Louis, Mo.). All other chemical reagents were purchased from Aldrich (Milwaukee, Wis.).

Bead library screenings were performed in disposable polypropylene columns from Perkin-Elmer Life Sciences. All buffer reagents were from Sigma unless otherwise noted. The anti-GST-alkaline phosphatase conjugate came from Rockland (Gilbertsville, Pa.). Mouse monoclonal anti-p21 antibody was obtained from Upstate Biotechnology Inc. (Lake Placid, N.Y.). Mouse anti-ubiquitin antibody, rabbit anti-p27 antibody, and rabbit anti-p57 antibody came from Santa Cruz Biotechnology Inc. (Santa Cruz, Calif.) Rabbit anti-human PARP antibody was obtained from Cell Signaling (Danvers, Mass.). Goat anti-mouse and goat anti-rabbit horseradish peroxidase-conjugated IgG were obtained from Bio-Rad (Richmond, Calif.). ECL Western Blotting Detection Reagents were obtained from Amersham Biosciences (Buckinghamshire, United Kingdom).

ACHN and A498 human RCC cells (from ATCC) were maintained in MEM 1× media supplemented with 10% fetal bovine serum, 2 mM L-glutamine, 1 mM sodium pyruvate, 0.1 mM non-essential amino acid, and 0.75% sodium bicarbonate at 37° C. in a humidified incubator containing 5% CO₂ in air.

### Example 1

### Synthesis of an Encoded Small Molecule OBOC Combinatorial Library

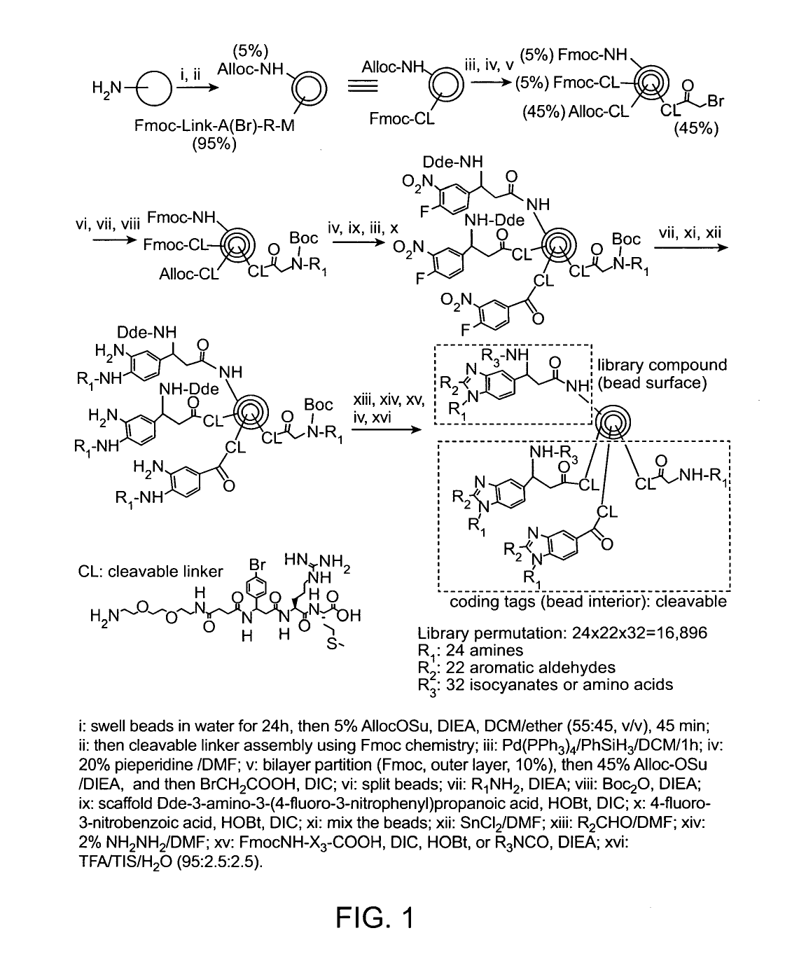

A small molecule OBOC combinatorial library was synthesized on bilayer TG beads (loading 0.26 mmol/g) using a similar approach as previously reported ((Liu R, Marik J, Lam K S. J Am Chem Soc 2002; 124(26):7678-7680; Liu R, Wang X, Song A, Bao T, Lam K S. QSAR & Combinatorial Science 2005; 24:1127-1140; Wang X, Zhang J, Song A, Lebrilla C B, Lam K S. J Am Chem Soc 2004; 126(18):5740-5749). The synthetic approach is shown in FIG. 1. Briefly, TG resin was first topologically segregated into two layers with an allyloxycarbonyl (Alloc)-protected outer layer (the thickness: 5% of the overall bead substitution) and a free N-terminal interior (thickness: 95% of the overall bead substitution) using the biphasic approach reported by Liu et al. (Liu R, Marik J, Lam K S. J Am Chem Soc 2002; 124(26):7678-7680). A cleavable linker (CL) was then assembled to the bead interior using 9-fluorenylmethyloxycarbonyl (Fmoc) peptide chemistry as reported by Wang et al. (Wang X, Zhang J, Song A, Lebrilla C B, Lam K S. J Am Chem Soc 2004; 126(18):5740-5749). After Fmoc deprotection, the beads were partitioned again with the same biphasic approach into two layers with different thickness, where the outer layer protected with Fmoc display two compounds (Fmoc-NH— on the bead surface and Fmoc-CL- in the middle), and the interior carry free N-terminus. The bead interior were partially protected with Alloc (50%) and then coupled with α-bromoacetic acid using DIC. The beads were evenly split into 24 aliquots and each aliquot reacted with a specific amine (R₁NH₂), followed by Boc₂O protection in the presence of N,N′-diisopropylethylamine (DIEA). Upon Fmoc deprotection, each aliquot of beads was acylated with a trifunctional scaffold, N-Dde-3-amino-2-(4-fluoro-3-nitrophenyl)propionic acid [Dde: 1-(4,4-dimethyl-2,6-dioxocyclohex-1-ylidene)ethyl)] using DIC/HOBt. With the further Alloc- deprotection using palladium chemistry, the liberated free amines on the bead interior were conjugated with a coding scaffold, 4-fluoro-3-nitrobenzoic acid. Then each aliquot of beads were incubated overnight with the corresponding primary amine (R₁NH₂) in the presence of DIEA. All aliquots of beads were mixed and treated with SnCl₂.H₂O (2M) solution in DMF for 4 hr. The derivatized beads were split again to 22 aliquots, each of which was incubated overnight with a different aromatic aldehyde to form a benzoimidazole heterocycle. All aliquots of beads were mixed. Upon Dde deprotection using 2% of hydrazine solution in DMF for 10 min, the beads were split again into 32 aliquots. Each aliquot of the beads received either an Fmoc-protected amino acid or isothiocyanate. All beads were combined, Fmoc-deprotected, and treated with trifluoroacetic acid (TFA)-based cleavage cocktail (TFA/tiisopropylsilane (TIS)/H₂O, 95:2.5:2.5) for 2.5 hr. After thorough washing with DCM, DMF, MeOH, H₂O and phosphate buffered seline (PBS) buffer, the library was ready for biological screening.

### Example 2

### Resynthesis of Compounds on TG Beads and in Soluble Form

Synthesis on TentaGel beads. By use of the similar synthetic approach described in the library synthesis except an Alloc-protected scaffold (instead of Dde-protected) is used, small molecules No. 1-12 (Table 1) were resynthesized on TG beads for re-binding assay. Briefly, TG resin was coupled with the scaffold and split into 12 same portions. And then each same volume of resin was coupled with the corresponding amines using DIEA as a base, followed by NO₂ reduction with 2 M SnCl₂.2H₂O solution in DMF. And then the corresponding aldehydes were coupled to the beads. The Alloc group on the scaffold was deprotected by Pd(PPh₃)₄/PhSiH₃ solution in DCM. These resins were coupled with the corresponding Fmoc-amino acids in presence of HOBt/DIC or isocyanates in presence of DIEA. For the amino acids at R₃ position, Fmoc group was deprotected by 20% piperidine in DMF, and the side chain protecting groups were removed by TFA (94%)/TIS(1%)/H₂O (5%).

Synthesis of compounds in soluble form. Small molecule compounds were also synthesized in soluble form for biological tests. In this case, the compounds were synthesized on Rink-amide resin using the scheme shown in FIG. 9. In brief, Rink amide MBHA resin (loading 0.59 mmol/g) was first swollen in DMF for 3 h, then Fmoc was removed with 20% piperidine (5 min, 15 min). Alloc-3-amino-3-(4-fluoro-3-nitrophenyl)propanoic acid (scaffold, 3 eq. to resin), HOBt (3 eq.) and DIC (3 eq.) were dissolved in DMF and added to the beads. The reaction conducted at room temperature for 4 h. Kaiser test was used to check the completion of the coupling. The beads were washed with DMF, MeOH, DMF. Then, primary amine R₁NH₂ (5 eq.) and DIEA (10 eq.) in DMF solution were added to the beads and rotated overnight. NO₂ reduction was achieved with 2 M SnCl₂.2H₂O solution in DMF for 2 h (twice). The beads were washed with DMF, MeOH, DMF again, then R₂CHO (5 eq.) in DMF was added to the beads and rotated at room temperature for 2 days. After complete washing with DMF, MeOH, DCM, the beads were treated with Pd(PPh₃)₄ (0.2 eq.) and PhSiH₃ (20 eq.) in DCM for 1 h to remove the Alloc group. A DMF solution of R₃NCO (5 eq.) in presence of DIEA (10 eq.) was added the beads and rotated for several hours. Kaiser test was used to check the completion of reaction. After the reaction was done, the beads were washed with DMF, MeOH, DCM, then were dried over vacuum. Then, a cleavage solution containing 95% TFA, 2.5% TIS and 2.5% water was added to the beads. After 2 h, the liquid was collected in a 10 mL-tube. After evaporation of TFA and the solvents, the concentrated cleavage product was precipitated with cold ether and purified by semipreparative reversed-phase high-performance liquid chromatography (RP-HPLC). The purity of the ligands was analyzed by analytical RP-HPLC on a Beckman System Gold HPLC system (Fullerton, Calif., USA).

The identity of compounds 4,7 and 10 was analyzed on a Thermo Fisher (San Jose, Calif.) model LCQ equipped with an electrospray source using standard conditions. Compound 4: M/Z 622.4. Compound 7: M/Z 580.35. Compound 10: M/Z 652.35. The structures of remaining compounds were confirmed with a MALDI-TOF MS (see Table 1).

### Example 3

### Identification of Compounds for Inhibition of p21

A two-step screening of the small molecule OBOC combinatorial library was performed using GST-tagged p21 recombinant protein (GST-p21) from ProSpec-Tany TechnoGene Ltd. (Rehovot, Israel) (FIG. 2). Screening was conducted in small disposable chromatography columns. Each screening used 25,000-30,000 beads that were first blocked with 0.1% BSA in PBS with 0.1% Tween 20 (PBST). In Stage 1 of the procedure, the beads were incubated with a 1:10,000 dilution of GST-p21, washed, incubated with anti-GST antibody conjugated with alkaline phosphatase (anti-GST-AP), washed, and incubated with the phosphatase substrate 5-bromo-4-chloro-3-indolyl-phosphate (BCIP). Beads became blue and were selected for next step. Stage 2 of the procedure was to remove the false positive beads that are interacting with GST and anti-GST-AP not p21. The beads from Stage 1 were incubated with 8 M guanidine-HCl for removing bound proteins and DMF for removing blue color followed by PBST washing and re-blocking with PBST. And then the beads were incubated with GST alone, washed, incubated with anti-GST-AP, washed, and incubated with the phosphatase substrate BCIP. From this step, the clear beads were considered true positive beads, therefore they were selected and deproteinated with 8 M guanidine-HCl prior to decoding with mass spectrometry. The coding tags on bead were cleaved off with cyanogen bromide in presence of TFA and analyzed by matrix-assisted laser desorption ionization time-of-flight mass spectrometry (MALDI-TOF MS) (Wang X, Zhang J, Song A, Lebrilla C B, Lam K S. J Am Chem Soc 2004; 126(18):5740-5749). The chemical structures of small molecule ligands were constructed based on the corresponding identity of the coding tags.

A 200 μL aliquot of cells (1×10³ cells in quiescent media) was added to a 96 well plate and incubated for 18 hr at 37° C. in a humidified incubator containing 5% CO₂ in air. After incubation, each condition of small molecule ligands (No. 4, 7, and 10) was added into each well for 48 hr and for synergic assay of Doxorubicin, after incubation for 6 hr with compound No. 10, Doxorubicin dissolved in DMSO was added to each of the 96 wells and incubated for 42 hr. Control cultures were treated with DMSO. After incubation, a 20 μL MTT solution (5 mg/mL in phosphate buffer) was added to each well and the incubation continued for 4 hr, after which time the solution in each well was carefully removed. The blue crystalline precipitate in each well was dissolved in DMSO 200 μL). The visible absorbance at 560 nm of each well was quantified using a microplate reader.

### Example 4

### Immunoblotting and Immunoprecipitation

Cells were washed with PBS and lysed in lysis buffer (50 mM HEPES, 1% Triton X-100, 10 mM sodium pyrophosphate, 100 mM sodium fluoride, 4 mM EDTA) at 0° C. Cell lysates were centrifuged (13,000×g, 4° C., 20 min) and the supernatants were electrophoresed and immunoblotted. For immunoprecipitation, cell lysates were incubated with p21 antibody agarose-conjugate overnight at 4° C. and immunoprecipitates were washed five times with lysis buffer. The bound proteins were eluted with SDS sample buffer and elctophoresed and immnoblotted. The membranes were blocked in 5% non-fat dry milk for 1 hr at room temperature, and probed with appropriate antibodies. Membranes were then probed with HRP-tagged anti-mouse or anti-rabbit IgG antibodies diluted 1:5,000-1:15,000 in 5% non-fat dry milk for 1 hr at room temperature. Chemiluminescence was detected using enhanced ECL.

### Example 5

### Treatment of Kidney Cancer

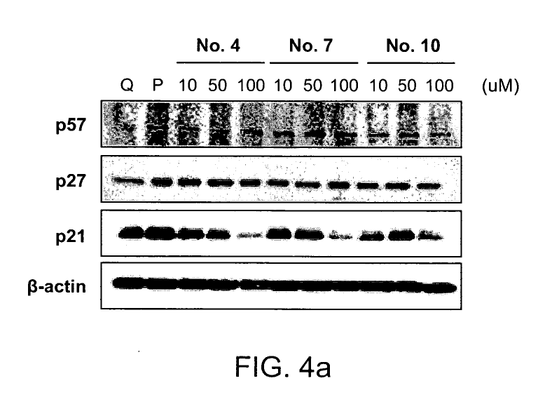

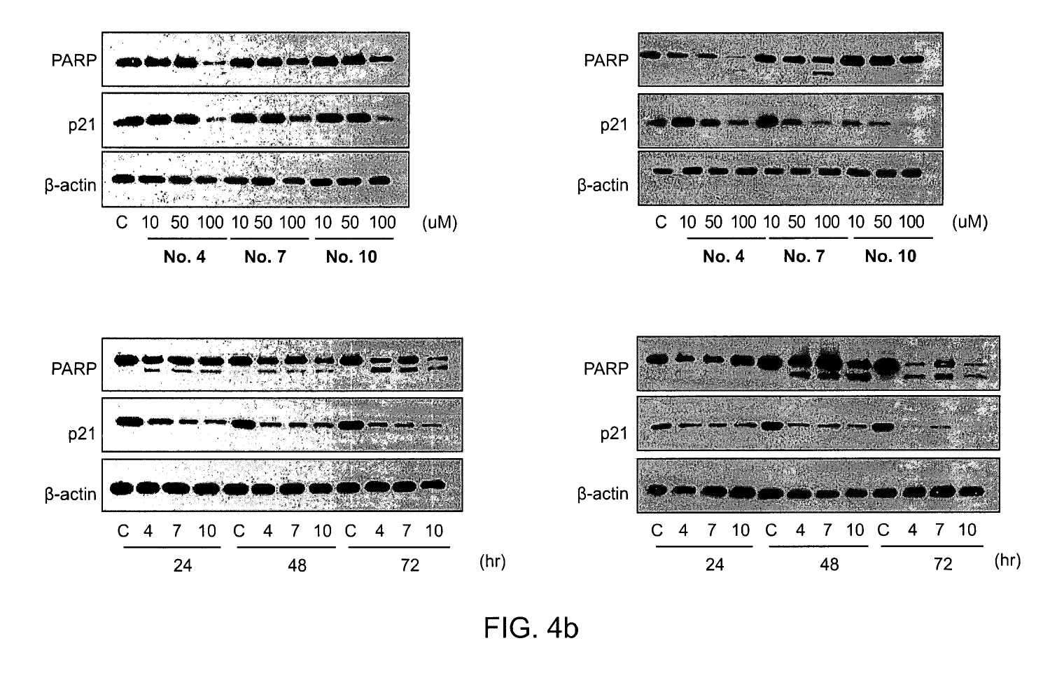

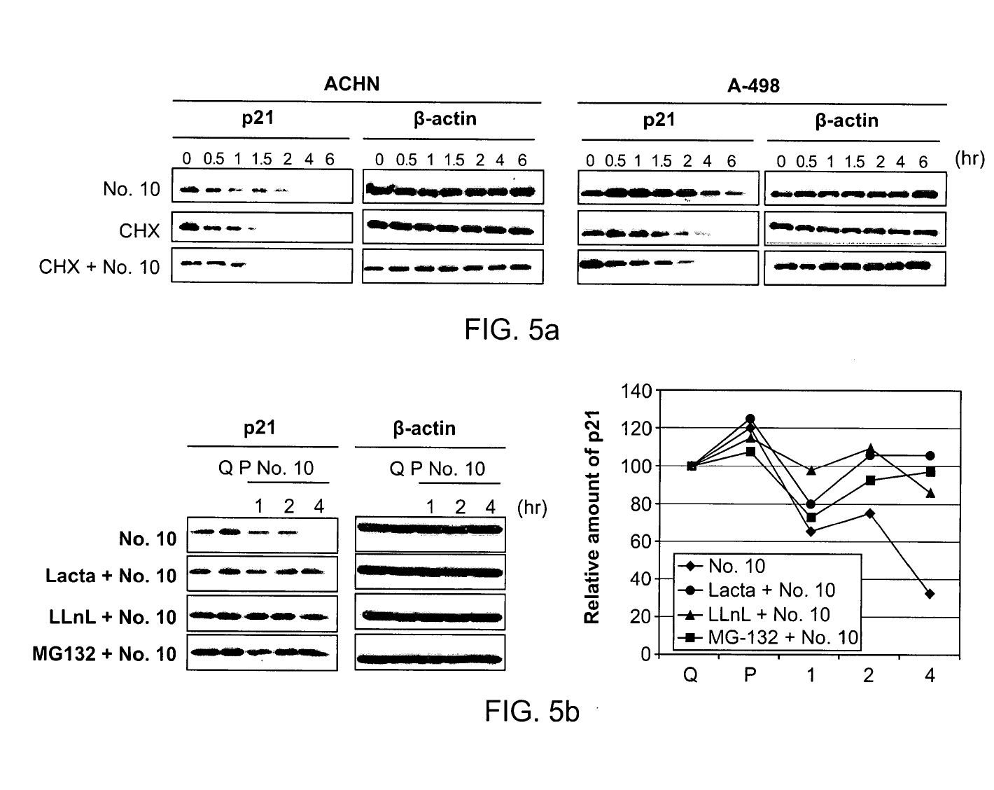

Compounds 4, 7, and 10, were incubated with serum-starved ACHN and A-498 cells for 48 hr and levels of p21, as well as assays of apoptosis and cell viability, were performed. p21 protein levels were markedly and specifically inhibited in both cell lines by all three compounds in a dose-dependent manner (FIG. 4a). Attenuation of p21 by these compounds was specific as there was no affect on levels of the other CKIs p27 and p57 at all concentrations examined (FIG. 4a). Apoptosis as measured by PARP cleavage increased, and cell viability as measured by MTT assay decreased, in parallel with p21 attenuation (FIG. 4b) for up to 72 h, consistent with the known anti-apoptotic effects of p21 in RCC as well as other cancer cells (Fan Y, Borowsky A D, Weiss R H. Mol Cancer Ther 2003; 2(8):773-782; Park S H, Park J Y, Weiss R H. J Urol 2008; 180(1):352-360; Gartel A L, Tyner A L. Mol Cancer Ther 2002; 1:639-649). Thus, the p21-binding compounds 4, 7, and 10 are pro-apoptotic in two RCC cell lines, by means of their specific attenuation of cellular p21 levels.

### Example 6

### Combination Therapy for the Treatment of Kidney Cancer

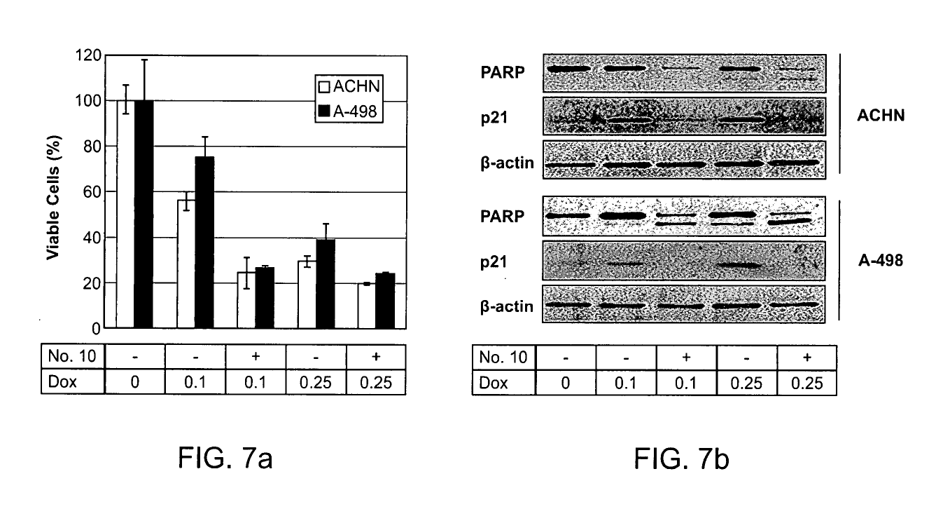

Incubation of ACHN and A498 with compound 10 in the presence or absence of doxorubicin shows a synergistic decrease in cell survival and increase in apoptosis (FIG. 7), demonstrating that these p21-binding compounds sensitize kidney cancer cells to DNA damaging chemotherapy and therefore have potential clinical use against a notoriously chemotherapy resistant cancer type.

Although the foregoing invention has been described in some detail by way of illustration and example for purposes of clarity of understanding, one of skill in the art will appreciate that certain changes and modifications can be practiced within the scope of the appended claims. In addition, each reference provided herein is incorporated by reference in its entirety to the same extent as if each reference was individually incorporated by reference.

What is claimed is:

1. A compound of formula I: 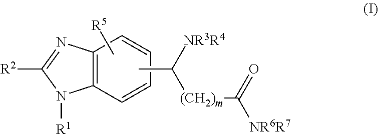
wherein
R¹ is selected from the group consisting of C₁₋₆ alkyl, C₀₋₆ alkyl-NR⁸R⁹, C₀₋₆ alkyl-cycloalkyl, C₀₋₆ alkyl-heterocycloalkyl, C₀₋₆ alkyl-aryl and C₀₋₆ alkyl-heteroaryl, each optionally substituted with from 1-4 R^{1a} groups;
each R^{1a} is independently selected from the group consisting of H, halogen, C₁₋₆ alkyl, C₁₋₆ haloalkyl, C₂₋₆ alkenyl, C₂₋₆ alkynyl, C₁₋₆ alkoxy, C₁₋₆ haloalkoxy, C₁₋₆ hydroxyalkyl, —OH, —C₀₋₆ alkyl-NR⁸R⁹, —SR⁸, —C(O)R⁸, —C₀₋₆ alkyl-C(O)OR⁸, —C(O)NR⁸R⁹, —N(R⁸)C(O)R⁹, —N(R⁸)C(O)OR⁹, —N(R⁸)C(O)NR⁸R⁹, —OP(O)(OR⁸)₂, —S(O)₂OR⁸, —S(O)₂NR⁸R⁹, —CN, C₀₋₆ alkyl-cycloalkyl, heterocycloalkyl, C₀₋₆ alkyl-aryl and heteroaryl, alternatively, two R^{1a} groups are joined to form ═O;
R² is selected from the group consisting of C₀₋₆ alkyl-aryl, C₂₋₆ alkenyl-aryl, C₀₋₆ alkyl-heteroaryl, and —N(R⁸)-aryl, each optionally substituted with 1-4 members each independently selected from the group consisting of R^{1a}, —OR¹⁰, —SR¹⁰ and —NR⁸R¹⁰;
R³ is selected from the group consisting of —C(O)—C₁₋₆ alkyl, —C(O)—C₁₋₆ hydroxyalkyl, —C(O)—C₁₋₆ alkylamine, —C(O)—heterocycloalkyl, —C(O)—NR^{3a}R^{3b}, —C(O)OR^{3a}, —S(O)₂R^{3a} and an amino acid;
each of R^{3a} and R^{3b} are independently selected from the group consisting of H, C₁₋₆ alkyl, cycloalkyl, heterocycloalkyl, aryl, and heteroaryl each optionally substituted with from 1-4 R⁸ groups;
R⁴, R⁵, R⁶, R⁷, R⁸ and R⁹ are each independently selected from the group consisting of H and C₁₋₆ alkyl;
R¹⁰ is selected from the group consisting of cycloalkyl, heterocycloalkyl, aryl and heteroaryl, each optionally substituted with from 1-4 R^{1a} groups;
subscript m is an integer from 0-2; and
salts, hydrates, prodrugs, and isomers thereof.

2. The compound of claim 1, having the following formula: 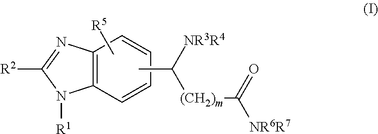
wherein
R¹ is selected from the group consisting of C₁₋₆ alkyl, C₀₋₆ alkyl-cycloalkyl, C₀₋₆ alkyl-heterocycloalkyl, C₀₋₆ alkyl-aryl and C₀₋₆ alkyl-heteroaryl, each optionally substituted with from 1-4 R^{1a} groups;
each R^{1a} is independently selected from the group consisting of H, halogen, C₁₋₆ alkyl, C₁₋₆ haloalkyl, C₂₋₆ alkenyl, C₂₋₆ alkynyl, C₁₋₆ alkoxy, C₁₋₆ haloalkoxy, C₁₋₆ hydroxyalkyl, —OH, —C₀₋₆ alkyl-NR⁸R⁹, —SR⁸, —C(O)R⁸, —C₀₋₆ alkyl-C(O)OR⁸, —C(O)NR⁸R⁹, —N(R⁸)C(O)R⁹, —N(R⁸)C(O)OR⁹, —N(R⁸)C(O)NR⁸R⁹, —OP(O)(OR⁸)₂, —S(O)₂OR⁸, —S(O)₂NR⁸R⁹, —CN, C₀₋₆ alkyl-cycloalkyl, heterocycloalkyl, C₀₋₆ alkyl-aryl and heteroaryl;
R² is selected from the group consisting of C₀₋₆ alkyl-aryl, C₂₋₆ alkenyl-aryl, and C₀₋₆ alkyl-heteroaryl, each optionally substituted with from 1-4 R^{1a} groups;
R³ is selected from the group consisting of —C(O)—C₁₋₆ alkyl, —C(O)—C₁₋₆ hydroxyalkyl, —C(O)—C₁₋₆ alkylamine, —C(O)-heterocycloalkyl, —C(O)—NR^{3a}R^{3b}, —C(O)OR^{3a}, —S(O)₂R^{3a} and an amino acid;
each of R^{3a} and R^{3b} are independently selected from the group consisting of H, C₁₋₆ alkyl, cycloalkyl, heterocycloalkyl, aryl, and heteroaryl each optionally substituted with from 1-4 R⁸ groups;
R⁴, R⁵, R⁶, R⁷, R⁸ and R⁹ are each independently selected from the group consisting of H and C₁₋₆ alkyl; and
subscript m is an integer from 0-2.

3. The compound of claim 1, wherein the compound is of formula Ia:

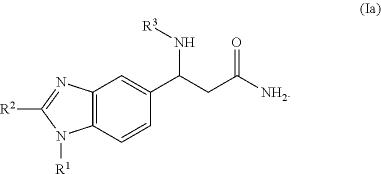

4. The compound of claim 3, wherein:
R¹ is selected from the group consisting of C₀₋₆ alkyl-cycloalkyl, and C₀₋₆ alkyl-heterocycloalkyl, each optionally substituted with a member selected from the group consisting of H, C₁₋₆ alkyl, and C₀₋₆ alkyl-aryl;
R² is selected from the group consisting of aryl, C₂₋₆ alkenyl-aryl, and heteroaryl, each optionally substituted with a member selected from the group consisting of H, halogen, C₁₋₆ alkyl, C₁₋₆ alkoxy, and —OH;
R³ is selected from the group consisting of —C(O)—C₁₋₆ alkyl, —C(O)—C₁₋₆ alkylamine, —C(O)—NR^{3a}R^{3b}, and an amino acid.

5. The compound of claim 4, wherein
R¹ is C₀₋₆ alkyl-piperidinyl;
R² is selected from the group consisting of phenyl and anthracenyl; and
R³ is —C(O)—NHR^{3a}, wherein R^{3a} is phenyl, optionally substituted with C₁₋₆ alkyl.

6. The compound of claim 3, wherein R¹ is

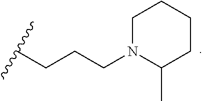

7. The compound of claim 3, wherein R² is

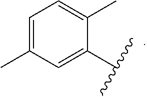

8. The compound of claim 3, wherein R³ is 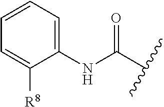
wherein R⁸ is C₁₋₆ alkyl.

9. The compound of claim 1, wherein the compound is selected from the group consisting of:

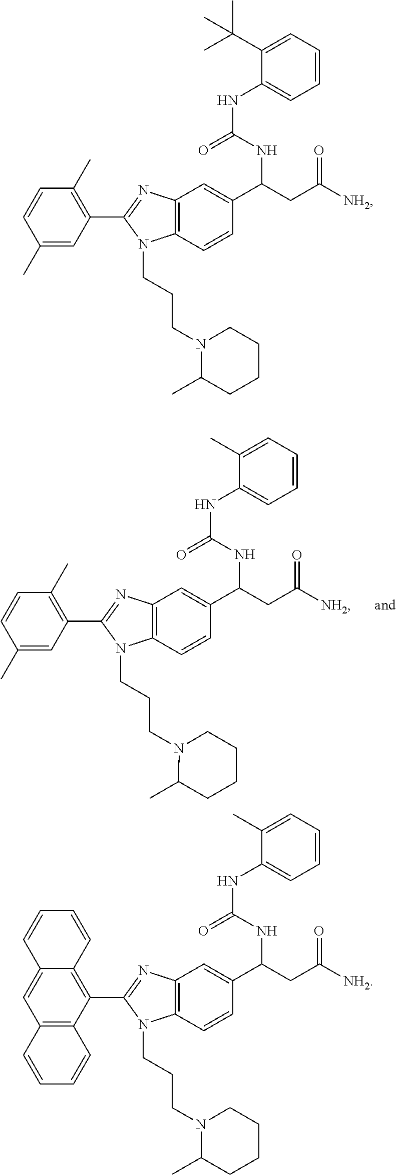

10. The compound of claim 3, wherein:
R¹ is selected from the group consisting of C₀₋₆ alkyl-NR⁸R⁹, C₀₋₆ alkyl-cycloalkyl, and C₀₋₆ alkyl-heterocycloalkyl, each optionally substituted with from 1-4 R^{1a} groups;
R² is selected from the group consisting of aryl, C₂₋₆ alkenyl-aryl, heteroaryl, aryl-O-aryl, and —NH-aryl, each optionally substituted with from 1-4 R^{1a} groups; and
R³ is selected from the group consisting of —C(O)—C₁₋₆ alkyl, —C(O)—C₁₋₆ alkylamine, —C(O)—NR^{3a}R^{3b}, and an amino acid.

11. The compound of claim 3, wherein
R¹ is selected from the group consisting of C₀₋₆ alkyl-piperidinyl and C₀₋₆ alkyl-NR⁸R⁹, each optionally substituted with from 1-4 R^{1a} groups;
R² is phenyl-O-phenyl, optionally substituted with from 1-4 R^{1a} groups; and
R³ is —C(O)—NHR^{3a}, wherein R^{3a} is phenyl, optionally substituted with C₁₋₆ alkyl.

12. The compound of claim 3, wherein R² is

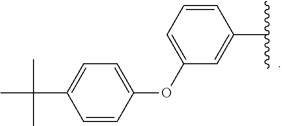

13. The compound of claim 3, wherein the compound has formula Ib:

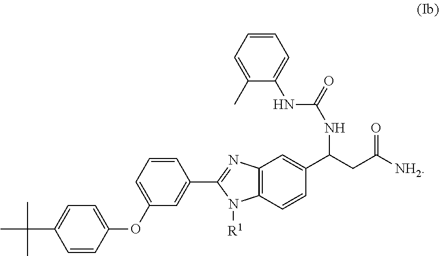

14. The compound of claim 1, selected from the group consisting of

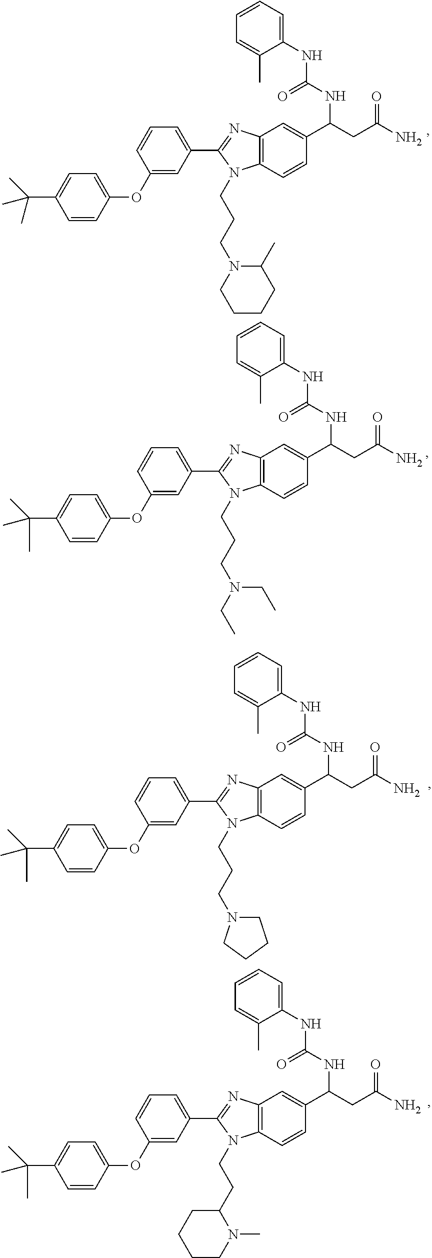

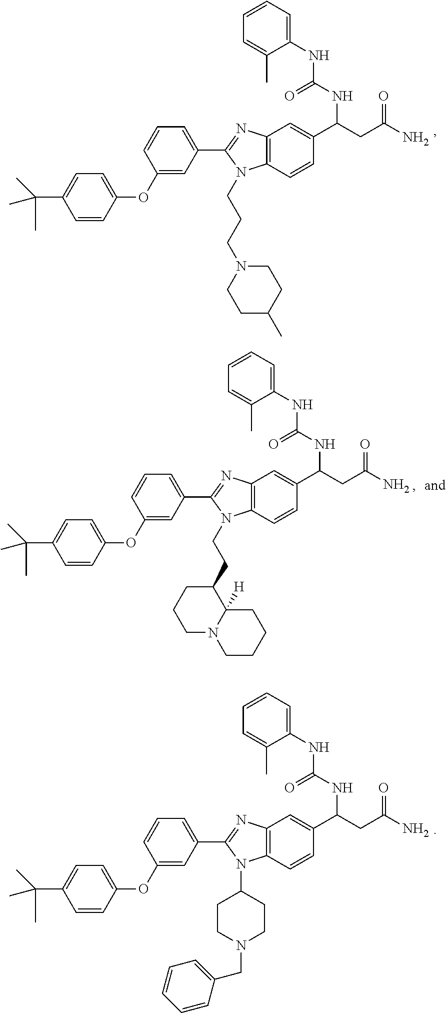

15. A pharmaceutical composition comprising a compound of claim 1 and a pharmaceutically acceptable excipient.

16. A method of inhibiting cyclin kinase inhibitor p21 comprising administering to a subject in need thereof a therapeutically effective amount of a compound of claim 1.

17. A method of treating cancer, the method comprising administering to a subject in need thereof a therapeutically effective amount of a compound of claim 1.

18. The method of claim 17, wherein the cancer is kidney cancer.
